SEQUENCE LISTING (8 sequences)

SEQ ID NO: 1 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 2 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 3 - protein, 73 aa, Homo sapiens
RQDPESLPFRQPVDPQLLGIPDYFDIVKNPMDLSTIKRKLDTGQYQEPWQYVDDVWLMFNNAWLYNRKTSRVY

SEQ ID NO: 4 - protein, 73 aa, Homo sapiens
RQDPESLPFRQPVDPQLLGIPDYFDIVKSPMDLSTIKRKLDTGQYQEPWQYVDDIWLMFNNAWLYNRKTSRVY

SEQ ID NO: 5 - protein, 116 aa, Homo sapiens
KKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLG

SEQ ID NO: 6 - protein, 122 aa, Homo sapiens
APGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLG

SEQ ID NO: 7 - protein, 380 aa, Homo sapiens
ENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 8 - protein, 381 aa, Homo sapiens
ENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQD

(19) United States
(12) Patent Application Publication
(10) Pub. No.: US 2016/0317632 A1
(43) Pub. Date: Nov. 3, 2016

(54) USE OF CBP/EP300 BROMODOMAIN INHIBITORS FOR CANCER IMMUNOTHERAPY

(71) Applicants: GENENTECH, INC., SOUTH SAN FRANCISCO, CA (US); CONSTELLATION PHARMACEUTICALS, INC., CAMBRIDGE, MA (US)

(72) Inventors: Brian K. Albrecht, Cambridge, MA (US); James Edmund Audia, Cambridge, MA (US); Steven Bellon, Cambridge, MA (US); Andrea Cochran, South San Francisco, CA (US); Alexandre Cote, Cambridge, MA (US); Terry Crawford, South San Francisco, CA (US); Benjamin Fauber, South San Francisco, CA (US); Srimoyee Ghosh, Cambridge, MA (US); Jean-Christophe Harmange, Cambridge, MA (US); Georgia Hatzivassiliou, South San Francisco, CA (US); Hariharan Jayaram, Cambridge, MA (US); Jeong Kim, South San Francisco, CA (US); Jose M. Lora, Cambridge, MA (US); Steven Magnuson, South San Francisco, CA (US); Ira Mellman, South San Francisco, CA (US); Anthony F. Romero, South San Francisco, CA (US); Alexander M. Taylor, Cambridge, MA (US); Vickie Tsui, South San Francisco, CA (US)

(73) Assignees: GENENTECH, INC., SOUTH SAN FRANCISCO, CA (US); CONSTELLATION PHARMACEUTICALS, INC., CAMBRIDGE, MA (US)

(21) Appl. No.: 15/095,985

(22) Filed: Apr. 11, 2016

Related U.S. Application Data
(63) Continuation of application No. PCT/US2014/060147, filed on Oct. 10, 2014.
(60) Provisional application No. 61/890,041, filed on Oct. 11, 2013.

(51) Int. Cl. A61K 39/00; A61K 31/498; G01N 33/50; A61K 31/538

(52) U.S. Cl. CPC A61K 39/0011; A61K 31/538; A61K 31/498; G01N 33/5011

(57) ABSTRACT

The present invention relates to use of CBP/EP300 bromodomain inhibitors for the treatment of cancer.

### CROSS REFERENCE TO RELATED APPLICATION

This application is a continuation of International Application No. PCT/US2014/060147, filed Oct. 10, 2014, which claims the benefit of and priority to U.S. Provisional Application Ser. No. 61/890,041, filed Oct. 11, 2013, which applications are incorporated by reference in their entirety.

### SEQUENCE LISTING

The instant application contains a Sequence Listing which has been submitted electronically in ASCII format and is hereby incorporated by reference in its entirety. Said ASCII copy, created on Oct. 10, 2014, is named 01075.004WO1_SL.txt and is 53,084 bytes in size.

### TECHNICAL FIELD

The present invention relates to use of CBP/EP300 bromodomain inhibitors for the treatment of cancer.

### BACKGROUND

Chromatin is a complex combination of DNA and protein that makes up chromosomes. It is found inside the nuclei of eukaryotic cells and is divided between heterochromatin (condensed) and euchromatin (extended) forms. The major components of chromatin are DNA and proteins. Histones are the chief protein components of chromatin, acting as spools around which DNA winds. The functions of chromatin are to package DNA into a smaller volume to fit in the cell, to strengthen the DNA to allow mitosis and meiosis, and to serve as a mechanism to control expression and DNA replication. The chromatin structure is controlled by a series of post-translational modifications to histone proteins, notably histones H3 and H4, and most commonly within the “histone tails” which extend beyond the core nucleosome structure. Histone tails tend to be free for protein-protein interaction and are also the portion of the histone most prone to post-translational modification. These modifications include acetylation, methylation, phosphorylation, ubiquitinylation, SUMOylation. These epigenetic marks are written and erased by specific enzymes that place the tags on specific residues within the histone tail, thereby forming an epigenetic code, which is then interpreted by the cell to allow gene specific regulation of chromatin structure and thereby transcription.

Of all classes of proteins, histones are amongst the most susceptible to post-translational modification. Histone modifications are dynamic, as they can be added or removed in response to specific stimuli, and these modifications direct both structural changes to chromatin and alterations in gene transcription. Distinct classes of enzymes, namely histone acetyltransferases (HATs) and histone deacetylases (HDACs), acetylate or de-acetylate specific histone lysine residues (Struhl K., Genes Dev., 1989, 12, 5, 599-606).

Covalent modification of histones is a fundamental mechanism of control of gene expression, and one of the major epigenetic mechanisms at play in eukaryotic cells (Kouzarides, Cell, 128, 693-705 (2007)). Because distinct transcriptional states define fundamental cellular processes, such as cell type specification, lineage commitment, cell activation and cell death, their aberrant regulation is at the core of a range of diseases (Medzhitov et al., Nat. Rev. Immunol., 9, 692-703 (2009); Portela et al., Nat. Biotech., 28, 1057-1068 (2010)). A fundamental component of the epigenetic control of gene expression is the interpretation of histone modifications by proteins that harbor specialized motifs that bind to such modifications. Among them, bromodomains have evolved to bind to acetylated histones and by so doing they represent fundamental links between chromatin structure and gene transcription (Fillipakoppoulos et al., Cell, 149, 214-231 (2012)).

Bromodomains, which are approximately 110 amino acids long, are found in a large number of chromatin-associated proteins and have been identified in approximately 70 human proteins, often adjacent to other protein motifs (Jeanmougin F., et al., Trends Biochem. Sci., 1997, 22, 5, 151-153; and Tamkun J. W., et al., Cell, 1992, 7, 3, 561-572). Interactions between bromodomains and modified histones may be an important mechanism underlying chromatin structural changes and gene regulation. Bromodomain-containing proteins have been implicated in disease processes including cancer, inflammation and viral replication. See, e.g., Prinjha et al., Trends Pharm. Sci., 33(3):146-153 (2012) and Muller et al., Expert Rev., 13(29):1-20 (September 2011).

Cell-type specificity and proper tissue functionality requires the tight control of distinct transcriptional programs that are intimately influenced by their environment. Alterations to this transcriptional homeostasis are directly associated with numerous disease states, most notably cancer, immuno-inflammation, neurological disorders, and metabolic diseases. Bromodomains reside within key chromatin modifying complexes that serve to control distinctive disease-associated transcriptional pathways. This is highlighted by the observation that mutations in bromodomain-containing proteins are linked to cancer, as well as immune and neurologic dysfunction. Hence, the selective inhibition of bromodomains across the family creates varied opportunities as novel therapeutic agents in human dysfunction.

There is a need for treatments for cancer, immunological disorders, and other bromodomain related diseases.

### SUMMARY

One aspect of the present invention is a method for treating cancer in an animal comprising administering an effective amount of a CBP/EP300 bromodomain inhibitor to the animal

One aspect of the present invention is a method for treating or delaying progression of cancer in an individual comprising administering an effective amount of a CBP/EP300 bromodomain inhibitor to the individual.

One aspect of the present invention is a method of enhancing immune function in an individual having cancer comprising administering an effective amount of a CBP/EP300 bromodomain inhibitor.

In certain embodiments, CD8 T cells in the individual have enhanced priming, activation, proliferation and/or cytolytic activity relative to prior to the administration of the CBP/EP300 bromodomain inhibitor.

In certain embodiments, the number of CD8 T cells is elevated relative to prior to administration of the CBP/EP300 bromodomain inhibitor.

In certain embodiments, the CD8 T cell is an antigen-specific CD8 T cell.

In certain embodiments, the cancer has elevated levels of T-cell infiltration.

In certain embodiments, the cancer is associated with increased intratumoral Treg cell density.

In certain embodiments, the cancer is selected from acoustic neuroma, acute leukemia, acute lymphocytic leukemia, acute myelocytic leukemia, acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer (NSCLC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

In certain embodiments, the cancer is melanoma, NSCLC, renal, ovarian, colon, pancreatic, hepatocellular, or breast cancer.

In certain embodiments, the cancer is NSCLC, ovarian, pancreatic, hepatocellular, or breast cancer.

In certain embodiments, the cancer is melanoma, NSCLC, or renal cell carcinoma.

In certain embodiments, the CBP/EP300 bromodomain inhibitor inhibits CBP.

In certain embodiments, the CBP/EP300 bromodomain inhibitor inhibits EP300.

In certain embodiments, the method suppresses Treg function.

In certain embodiments, the method decreases T cell exhaustion of CD8⁺ T cells.

In certain embodiments, the CBP/EP300 bromodomain inhibitor does not bind to the HAT domain of CBP and/or EP300.

In certain embodiments, the individual is a human, e.g., a female or male.

One aspect of the present invention a CBP/EP300 bromodomain inhibitor for use in medical treatment or diagnosis including therapy and/or treating cancer.

One aspect of the present invention is a method for selecting an anti-cancer compound, comprising determining whether a test compound is a CBP/EP300 bromodomain inhibitor compound, wherein a test compound that is a CBP/EP300 bromodomain inhibitor compound is selected as an anti-cancer compound.

In certain embodiments, the methods disclosed herein further comprise, determining whether the test compound binds to the HAT domain of CBP and/or EP300, wherein a test compound that does not bind to the HAT domain of CBP and/or EP300 is selected as an anti-cancer compound.

In certain embodiments, the method further comprises determining whether the test compound suppresses Treg function, wherein a test compound that suppresses Treg function is selected as an anti-cancer compound.

In certain embodiments, the method further comprises determining whether the test compound decreases T cell exhaustion of CD8⁺ T cells, wherein a test compound that decreases T cell exhaustion of CD8⁺ T cells is selected as an anti-cancer compound.

In certain embodiments, the CBP/EP300 bromodomain inhibitor compounds may include compounds of Formula I, an isomer or a mixture of isomers thereof (e.g., enantiomers) or a pharmaceutically acceptable salt, solvate or prodrug thereof. Such compounds, and processes and intermediates that are useful for preparing such compounds, are described in Angew. Chem. Int. Ed., 2014, v53, pages 1-6 and corresponding supporting information. In some embodiments, the compounds of Formula I include:

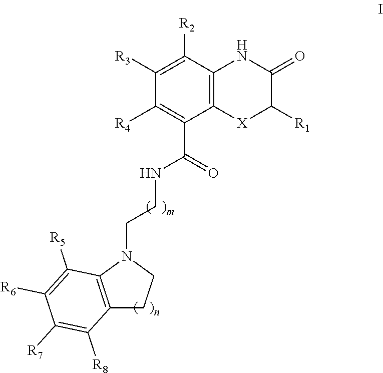

wherein:

X is NH or O;

m is 1 or 2;

n is 1 or 2;

R₁ is independently selected from the group consisting of substituted or unsubstituted C₁-C₆ alkyl, substituted or unsubstituted C₂₋₆ alkenyl, substituted or unsubstituted C₂₋₆ alkynyl, and substituted or unsubstituted C₃₋₆carbocyclyl;

R₂ is independently selected from the group consisting of hydrogen, halogen, substituted or unsubstituted C₁-C₆ alkyl, substituted or unsubstituted C₂₋₆ alkenyl, and substituted or unsubstituted C₂₋₆ alkynyl;

R₃ independently selected from the group consisting of hydrogen, halogen, substituted or unsubstituted C₁-C₆ alkyl, substituted or unsubstituted C₂₋₆ alkenyl, and substituted or unsubstituted C₂₋₆alkynyl;

R₄ independently selected from the group consisting of hydrogen, halogen, substituted or unsubstituted C₁-C₆ alkyl, substituted or unsubstituted C₂₋₆alkenyl, and substituted or unsubstituted C₂₋₆ alkynyl;

R₅ independently selected from the group consisting of hydrogen, halogen, substituted or unsubstituted C₁-C₆ alkyl, substituted or unsubstituted C₂₋₆alkenyl, substituted or unsubstituted C₂₋₆alkynyl, and OC₁-C₆ alkyl;

R₆ independently selected from the group consisting of hydrogen, halogen, substituted or unsubstituted C₁-C₆ alkyl, substituted or unsubstituted C₂₋₆ alkenyl, substituted or unsubstituted C₂₋₆alkynyl, and OC₁-C₆ alkyl;

R₇ independently selected from the group consisting of hydrogen, halogen, substituted or unsubstituted C₁-C₆ alkyl, substituted or unsubstituted C₂₋₆ alkenyl, substituted or unsubstituted C₂₋₆alkynyl, and OC₁-C₆ alkyl; and

R₈ independently selected from the group consisting of hydrogen, halogen, substituted or unsubstituted C₁-C₆ alkyl, substituted or unsubstituted C₂₋₆ alkenyl, substituted or unsubstituted C₂₋₆alkynyl, and OC₁-C₆ alkyl;

or a salt thereof.

In certain embodiments, the compound of Formula I is selected from the group consisting of:

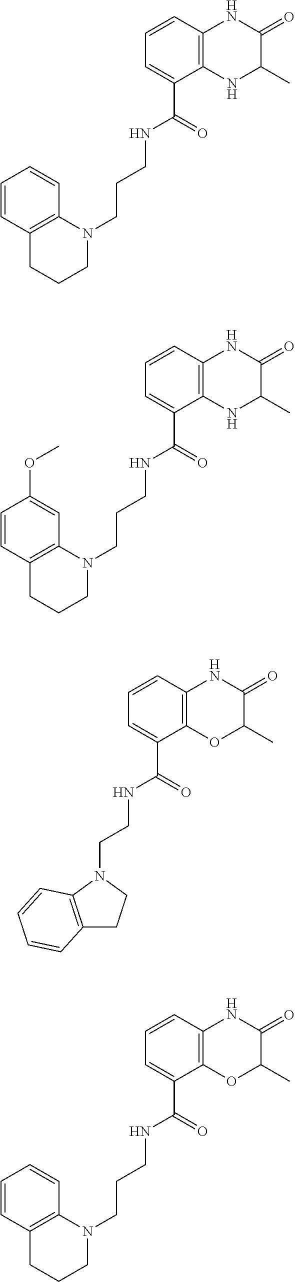 [SMILES: CC1Nc2c(cccc2C(=O)NCCCN2CCCc3ccccc32)NC1=O ; COc1ccc2c(c1)N(CCCNC(=O)c1cccc3c1NC(C)C(=O)N3)CCC2 ; CC1Oc2c(cccc2C(=O)NCCN2CCc3ccccc32)NC1=O ; CC1Oc2c(cccc2C(=O)NCCCN2CCCc3ccccc32)NC1=O]

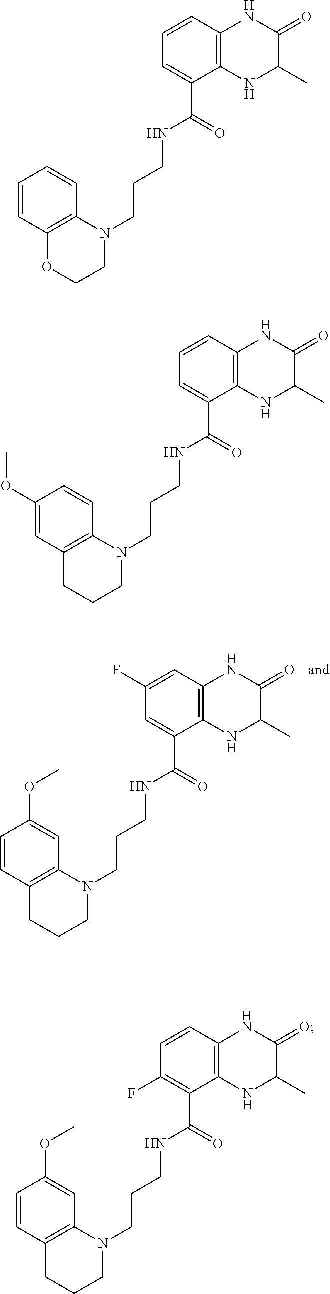 [SMILES: CC1Nc2c(cccc2C(=O)NCCCN2CCOc3ccccc32)NC1=O ; COc1ccc2c(c1)CCCN2CCCNC(=O)c1cccc2c1NC(C)C(=O)N2 ; COc1ccc2c(c1)N(CCCNC(=O)c1cc(F)cc3c1NC(C)C(=O)N3)CCC2 ; COc1ccc2c(c1)N(CCCNC(=O)c1c(F)ccc3c1NC(C)C(=O)N3)CCC2]

or a salt thereof.

### BRIEF DESCRIPTION OF THE FIGURES

FIG. 1. Human naïve T cells were cultured under Treg-differentiating conditions in the presence of active compound targeting the bromodomains of CBP/EP300, or inactive control compound. As depicted in FIG. 1, the CBP/EP300 inhibitor CBP/EP300(1), but not the inactive compound CBP/EP300(A), reduced the number of FOXP3+ cells generated, as seen by flow cytometry.

FIG. 2. Dose-response curves were determined with two exemplar active compounds from distinct chemical scaffolds, CBP/EP300(1) and CBP/EP300(2). These active compounds, but not the inactive ones, CBP/EP300(A) and CBP/EP300(B), reduced the number of FOXP3+ cells in a dose-dependent manner (FIG. 2, upper panels). The activation marker CD25 was not affected by any compound treatment, suggesting that these cells are functional, although unable to differentiate into the Treg lineage (FIG. 2, lower panels).

FIG. 3. As shown in FIG. 3 (upper panels), incubation of human CD8 cells with CBP/EP300(1), but not with the inactive compound, CBP/EP300(A), resulted in a dose-dependent reduction in the expression of LAG3, TIM3 and CTLA4. CBP/EP300 bromodomain inhibition with CBP/EP300(1) did not affect effector function in CD8 cells, as the genes encoding Perforin, Granzyme B and EOMES (FIG. 3, lower panels) were not significantly changed upon compound treatment.

FIG. 4. As depicted in FIG. 4, production of the effector cytokines IFN-γ and TNFα were not affected by compound treatment.

FIG. 5. Proliferation of naïve T cells was monitored by FACS-based quantification of the dye. As shown in FIG. 5, ˜50% of naïve T cells were able to proliferate upon CD3/CD28 stimulation in the absence of Treg cells. However, when naïve T cells were combined with Treg cells, less than 10% were able to proliferate. Incubation with CBP/EP300(1) resulted in a dose-dependent inhibition of the Treg suppressive capacity, as seen by a corresponding increase in the percentage of naïve T cells able to proliferate. The inactive compound, CBP/EP300(A) had no impact, demonstrating specificity.

FIG. 6. Generation and regulation of antitumor immunity. Tumor cells can evade multiple immune checkpoints, and an aim of the immunotherapy described herein is to re-empower the immune system against cancer cells. (see, e.g., Mellman et al., Nature, 480, 480 (2011)).

FIG. 7. CBP inhibitors CBP/EP300(3) and CBP/EP300(4) decrease Foxp3 expression in iTreg cells in a dose dependent manner. Data show Foxp3 expression in iTreg differentiating cells, fold change over unstimulated naïve T cells.

FIG. 8. CBP inhibitors CBP/EP300(3) and CBP/EP300(4) decrease Foxp3 protein expression in iTreg cells. Data show flow cytometric zebra plots of Foxp3 expression using iTreg differentiating cells treated with DMSO alone as control (A), and different concentrations of CBP/EP300(4) (B) or CBP/EP300(3) (C), 4 days after stimulation.

FIG. 9. CBP inhibitors resulted in a dose-dependent reduction of in the expression of Lag3, CTLA4 and TIM3. Data show Lag3, CTLA4 and TIM3 expression in stimulated CD8+ T cells, fold change over unstimulated CD8+ T cells.

FIG. 10. CBP inhibitors CBP/EP300(3) and CBP/EP300(4) did not affect effector function of CD8+ T cells. Data show GZMB expression in stimulated CD8+ T cells, fold change over unstimulated CD8+ T cells.

### DETAILED DESCRIPTION

The present invention is concerned with methods of treating and/or delaying progression of cancer by pharmacologically interfering with a bromodomain harbored in one or more of the following proteins, CBP and/or EP300, also described herein as CBP/EP300. Embodiments of the present invention relate to the manipulation of the human immune system to target and eliminate/reduce the number of cancer cells, hereafter described as cancer immunotherapy. The discoveries described herein focus in particular on two subsets of T lymphocytes, namely regulatory CD4+ T cells, hereafter described as Treg cells, and CD8+ cytotoxic T cells, hereafter described as CD8 cells, as these cells are recognized as key mediators of the immune system's anti-tumor activity. As such, certain embodiments of the invention provide a CBP/EP300 bromodomain inhibitor for use in the prophylactic or therapeutic treatment of cancer.

### DEFINITIONS

As used herein, the term “CBP/EP300 bromodomain inhibitor” refers to a compound that binds to the CBP bromodomain and/or EP300 bromodomain and inhibits and/or reduces a biological activity of CBP and/or EP300. In some embodiments, CBP/EP300 bromodomain inhibitor binds to the CBP and/or EP300 primarily (e.g., solely) through contacts and/or interactions with the CBP bromodomain and/or EP300 bromodomain. In some embodiments, CBP/EP300 bromodomain inhibitor binds to the CBP and/or EP300 through contacts and/or interactions with the CBP bromodomain and/or EP300 bromodomain as well as additional CBP and/or EP300 residues and/or domains. In some embodiments, CBP/EP300 bromodomain inhibitor substantially or completely inhibits the biological activity of the CBP and/or EP300. In some embodiments, the biological activity is binding of the bromodomain of CBP and/or EP300 to chromatin (e.g., histones associated with DNA) and/or another acetylated protein. In certain embodiments, an inhibitor has an IC₅₀ or binding constant of less about 50 μM, less than about 1 μM, less than about 500 nM, less than about 100 nM, or less than about 10 nM. In some embodiments, the CBP/EP300 bromodomain inhibitor blocks CBP/EP300 activity so as to restore a functional response by T-cells (e.g., proliferation, cytokine production, target cell killing) from a dysfunctional state to antigen stimulation. In some embodiments, the CBP/EP300 bromodomain inhibitor binds to and inhibits CBP bromodomain. In some embodiments, the CBP/EP300 bromodomain inhibitor binds to and inhibits EP300 bromodomain.

The terms “CBP” and “CREB binding protein,” as used herein, refers to any native CBP from any vertebrate source, including mammals such as primates (e.g. humans) and rodents (e.g., mice and rats), unless otherwise indicated. The term encompasses “full-length,” unprocessed CBP as well as any form of CBP that results from processing in the cell. The term also encompasses naturally occurring variants of CBP, e.g., splice variants or allelic variants. In some embodiments, the amino acid sequence of an exemplary human CBP is UNIPROT Q92793-1. In some embodiments, the amino acid sequence of an exemplary human CBP is UNIPROT Q92793-2. In some embodiments, the amino acid sequence of an exemplary human CBP is shown in SEQ ID NO: 1.

The terms “EP300” and “E1A binding protein p300,” as used herein, refers to any native EP300 from any vertebrate source, including mammals such as primates (e.g. humans) and rodents (e.g., mice and rats), unless otherwise indicated. The term encompasses “full-length,” unprocessed EP300 as well as any form of EP300 that results from processing in the cell. The term also encompasses naturally occurring variants of EP300, e.g., splice variants or allelic variants. In some embodiments, the amino acid sequence of an exemplary human EP300 is UNIPROT Q09472. In some embodiments, the amino acid sequence of an exemplary human EP300 is shown in SEQ ID NO:2.

The terms “measurable affinity” and “measurably inhibit,” as used herein, refer to a measurable reduction in activity of a bromodomain between: (i) a sample comprising a CBP/EP300 bromodomain inhibitor or composition thereof and such bromodomain, and (ii) an equivalent sample comprising such bromodomain, in the absence of said compound, or composition thereof.

“Pharmaceutically acceptable salts” include both acid and base addition salts. It is to be understood that when a compound or Example herein is shown as a specific salt, the corresponding free-base, as well as other salts of the corresponding free-base (including pharmaceutically acceptable salts of the corresponding free-base) are contemplated.

“Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, benzenesulfonic acid, p-toluenesulfonic acid, salicyclic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particular organic non-toxic bases are isopropylamine, diethylamine, ethanolamine, tromethamine, dicyclohexylamine, choline, and caffeine.

A “solvate” refers to an association or complex of one or more solvent molecules and a compound of the present invention. Examples of solvents include water, isopropanol, ethanol, methanol, DMSO, ethyl acetate, acetic acid and ethanolamine. The term “hydrate” refers to the complex where the solvent molecule is water.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The phrase “substantially similar,” as used herein, refers to a sufficiently high degree of similarity between two numeric values (generally one associated with a molecule and the other associated with a reference/comparator molecule) such that one of skill in the art would consider the difference between the two values to not be of statistical significance within the context of the biological characteristic measured by said values (e.g., Kd values). The difference between said two values may be, for example, less than about 20%, less than about 10%, and/or less than about 5% as a function of the reference/comparator value. The phrase “substantially normal” refers to substantially similar to a reference (e.g., normal reference).

The phrase “substantially different,” refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values may be, for example, greater than about 10%, greater than about 20%, greater than about 30%, greater than about 40%, and/or greater than about 50% as a function of the value for the reference/comparator molecule.

The “presence,” “amount,” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., housekeeping biomarker).

“Reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., housekeeping biomarker).

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, e.g., a thin slice of tissue or cells cut from a tissue sample. It is understood that multiple sections of tissue samples may be taken and subjected to analysis, provided that it is understood that the same section of tissue sample may be analyzed at both morphological and molecular levels, or analyzed with respect to both polypeptides and polynucleotides.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polynucleotide analysis or protocol, one may use the results of the polynucleotide expression analysis or protocol to determine whether a specific therapeutic regimen should be performed.

An “effective amount” of an agent, e.g., a pharmaceutical formulation, refers to an amount effective, at dosages and for periods of time necessary, to achieve the desired therapeutic or prophylactic result. In some embodiments, the effective amount refers to an amount of a CBP/EP300 bromodomain inhibitor that (i) treats the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. In some embodiments, the effective amount of the CBP/EP300 bromodomain inhibitor may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. For cancer therapy, efficacy can, for example, be measured by assessing the time to disease progression (TTP) and/or determining the response rate (RR). In the case of immunological disorders, the therapeutic effective amount is an amount sufficient to decrease or alleviate an allergic disorder, the symptoms of an autoimmune and/or inflammatory disease, or the symptoms of an acute inflammatory reaction (e.g. asthma). In some embodiments, an effective amount is an amount of a chemical entity described herein sufficient to significantly decrease the activity or number of drug tolerant or drug tolerant persisting cancer cells.

The term “dysfunction” in the context of immune dysfunction, refers to a state of reduced immune responsiveness to antigenic stimulation. The term includes the common elements of both exhaustion and/or anergy in which antigen recognition may occur, but the ensuing immune response is ineffective to control infection or tumor growth.

The term “dysfunctional”, as used herein, also includes refractory or unresponsive to antigen recognition, specifically, impaired capacity to translate antigen recognition into downstream T-cell effector functions, such as proliferation, cytokine production (e.g., IL-2) and/or target cell killing.

The term “anergy” refers to the state of unresponsiveness to antigen stimulation resulting from incomplete or insufficient signals delivered through the T-cell receptor (e.g. increase in intracellular Ca⁺² in the absence of ras-activation). T cell anergy can also result upon stimulation with antigen in the absence of co-stimulation, resulting in the cell becoming refractory to subsequent activation by the antigen even in the context of costimulation. The unresponsive state can often be overriden by the presence of Interleukin-2. Anergic T-cells do not undergo clonal expansion and/or acquire effector functions.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors. Exhaustion can result from both extrinsic negative regulatory pathways (e.g., immunoregulatory cytokines) as well as cell intrinsic negative regulatory (costimulatory) pathways (PD-1, B7-H3, B7-H4, etc.).

“Enhancing T-cell function” means to induce, cause or stimulate a T-cell to have a sustained or amplified biological function, or renew or reactivate exhausted or inactive T-cells. Examples of enhancing T-cell function include: increased secretion of γ-interferon from CD8⁺ T-cells, increased proliferation, increased antigen responsiveness (e.g., clearance) relative to such levels before the intervention. In one embodiment, the level of enhancement is as least 50%, alternatively 60%, 70%, 80%, 90%, 100%, 120%, 150%, 200%. The manner of measuring this enhancement is known to one of ordinary skill in the art.

A “T cell dysfunctional disorder” is a disorder or condition of T-cells characterized by decreased responsiveness to antigenic stimulation. In a particular embodiment, a T-cell dysfunctional disorder is a disorder that is specifically associated with inappropriate CBP and/or EP300 activity. In another embodiment, T-cell dysfunctional disorder is one in which T-cells are anergic or have decreased ability to secrete cytokines proliferate, or execute cytolytic activity. In a specific aspect, the decreased responsiveness results in ineffective control of a pathogen or tumor expressing an immunogen. Examples of T cell dysfunctional disorders characterized by T-cell dysfunction include tumor immunity.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Immunogenicity” refers to the ability of a particular substance to provoke an immune response. Tumors are immunogenic and enhancing tumor immunogenicity aids in the clearance of the tumor cells by the immune response.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

“Treatment” (and variations such as “treat” or “treating”) refers to clinical intervention in an attempt to alter the natural course of the individual or cell being treated, and can be performed either for prophylaxis or during the course of clinical pathology. Desirable effects of treatment include one or more of preventing occurrence or recurrence of disease, alleviation of symptoms, diminishment of any direct or indirect pathological consequences of the disease, stabilized (i.e., not worsening) state of disease, preventing metastasis, decreasing the rate of disease progression, amelioration or palliation of the disease state, prolonging survival as compared to expected survival if not receiving treatment and remission or improved prognosis. In certain embodiments, a CBP/EP300 bromodomain inhibitor is used to delay development of a disease or disorder or to slow the progression of a disease or disorder. Those individuals in need of treatment include those already with the condition or disorder as well as those prone to have the condition or disorder, (for example, through a genetic mutation or aberrant expression of a gene or protein) or those in which the condition or disorder is to be prevented.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

The term “patient” or “individual” as used herein, refers to an animal, such as a mammal, such as a human. In one embodiment, patient or individual refers to a human.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signalling inhibitors; HDAC inhibitors, proteasome inhibitors; and inhibitors of cancer metabolism.

In one embodiment the cytotoxic agent is selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signalling inhibitors, HDAC inhibitors, proteasome inhibitors, and inhibitors of cancer metabolism. In one embodiment the cytotoxic agent is a taxane. In one embodiment the taxane is paclitaxel or docetaxel. In one embodiment the cytotoxic agent is a platinum agent. In one embodiment the cytotoxic agent is an antagonist of EGFR. In one embodiment the antagonist of EGFR is N-(3-ethynylphenyl)-6,7-bis(2-methoxyethoxy)quinazolin-4-amine (e.g., erlotinib). In one embodiment the cytotoxic agent is a RAF inhibitor. In one embodiment, the RAF inhibitor is a BRAF and/or CRAF inhibitor. In one embodiment the RAF inhibitor is vemurafenib. In one embodiment the cytotoxic agent is a PI3K inhibitor.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁ λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (M-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®);

and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

The term “PD-1 axis binding antagonist” is a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonists” is a molecule that decreases, blocks, inhibits, abrogates or interferes with signal transduction resulting from the interaction of PD-1 with one or more of its binding partners, such as PDL1, PDL2. In some embodiments, the PD-1 binding antagonist is a molecule that inhibits the binding of PD-1 to its binding partners. In a specific aspect, the PD-1 binding antagonist inhibits the binding of PD-1 to PDL1 and/or PDL2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PDL1 and/or PDL2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is nivolumab described herein (also known as MDX-1106-04, MDX-1106, ONO-4538, BMS-936558, and OPDIVO®). In another specific aspect, a PD-1 binding antagonist is pembrolizumab described herein (also known as MK-3475, Merck 3475, KEYTRUDA®, and SCH-900475). In another specific aspect, a PD-1 binding antagonist is CT-011 described herein (also known as hBAT or hBAT-1). In yet another specific aspect, a PD-1 binding antagonist is AMP-224 (also known as B7-DCIg) described herein.

The term “PDL1 binding antagonists” is a molecule that decreases, blocks, inhibits, abrogates or interferes with signal transduction resulting from the interaction of PDL1 with either one or more of its binding partners, such as PD-1, B7-1. In some embodiments, a PDL1 binding antagonist is a molecule that inhibits the binding of PDL1 to its binding partners. In a specific aspect, the PDL1 binding antagonist inhibits binding of PDL1 to PD-1 and/or B7-1. In some embodiments, the PDL1 binding antagonists include anti-PDL1 antibodies, antigen binding fragments thereof, immunoadhesins, fusion proteins, oligopeptides and other molecules that decrease, block, inhibit, abrogate or interfere with signal transduction resulting from the interaction of PDL1 with one or more of its binding partners, such as PD-1, B7-1. In one embodiment, a PDL1 binding antagonist reduces the negative co-stimulatory signal mediated by or through cell surface proteins expressed on T lymphocytes mediated signaling through PDL1 so as to render a dysfunctional T-cell less dysfunctional (e.g., enhancing effector responses to antigen recognition). In some embodiments, a PDL1 binding antagonist is an anti-PDL1 antibody. In a specific aspect, an anti-PDL1 antibody is YW243.55.S70 described herein. In another specific aspect, an anti-PDL1 antibody is MDX-1105 described herein (also known as BMS-936559). In still another specific aspect, an anti-PDL1 antibody is MPDL3280A described herein. In still another specific aspect, an anti-PDL1 antibody is MEDI4736 described herein.

The term “PDL2 binding antagonists” is a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

Recitation of ranges of values herein are merely intended to serve as a shorthand method of referring individually to each separate value falling within the range, unless otherwise indicated herein, and each separate value is incorporated into the specification as if it were individually recited herein.

As is understood by one skilled in the art, reference to “about” a value or parameter herein includes (and describes) embodiments that are directed to that value or parameter per se. For example, description referring to “about X” includes description of “X”.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. It is understood that aspect and embodiments of the invention described herein include “consisting” and/or “consisting essentially of” aspects and embodiments.

Uses of CBP/EP300 Bromodomain Inhibitors

Provided herein are methods of using a CBP/EP300 bromodomain inhibitor for the inhibition of a CBP/EP300 bromodomain (in vitro or in vivo). For example, provided herein are methods for treating a bromodomain-mediated disorder in an individual comprising administering a CBP/EP300 bromodomain inhibitor to the individual. In some embodiments, the bromodomain-mediated disorder is cancer.

Provided herein are methods for treating or delaying progression of cancer in an individual comprising administering to the individual an effective amount of a CBP/EP300 bromodomain inhibitor. In some embodiments, the CBP/EP300 bromodomain inhibitor binds to a bromodomain of CBP. In some embodiments, the CBP/EP300 bromodomain inhibitor binds to one or more residues of SEQ ID NO:5 (amino acid residues 1082-1197 of UniProt No. Q9279). In some embodiments, the CBP/EP300 bromodomain inhibitor binds to one or more residues of the amino acid sequence SEQ ID NO:3 (amino acid residues 1103-1175 of UniProt No. Q92793). In some embodiments, the CBP/EP300 bromodomain inhibitor binds to a bromodomain of EP300. In some embodiments, the CBP/EP300 bromodomain inhibitor binds to one or more residues of the amino acid sequence SEQ ID NO:6 (amino acid residues 1040-1161 of UniProt No. Q09472). In some embodiments, the CBP/EP300 bromodomain inhibitor binds to one or more residues of the amino acid sequence SEQ ID NO:4 (amino acid residues 1067-1139 of UniProt No. Q09472). In some embodiments, the CBP/EP300 bromodomain inhibitor binds to the bromodomain of EP300 and the bromodomain of CBP. In some embodiments, the CBP/EP300 bromodomain inhibitor binds SEQ ID NO:5 and SEQ ID NO:6. In some embodiments, the CBP/EP300 bromodomain inhibitor binds SEQ ID NO:3 and SEQ ID NO:4. In some embodiments, the CBP/EP300 bromodomain inhibitor inhibits and/or reduces binding of the CBP/EP300 bromodomain to chromatin. In some embodiments, the CBP/EP300 bromodomain inhibitor does not inhibit histone acetyl transferase activity of CBP/EP300.

Further, provided herein are methods of enhancing immune function in an individual having cancer comprising administering an effective amount of a CBP/EP300 bromodomain inhibitor. In some embodiments, the CBP/EP300 bromodomain inhibitor binds to a bromodomain of CBP. In some embodiments, the CBP/EP300 bromodomain inhibitor binds to one or more residues of the amino acid sequence of SEQ ID NO:3. In some embodiments, the CBP/EP300 bromodomain inhibitor binds to one or more residues of the amino acid sequence of SEQ ID NO:5. In some embodiments, the CBP/EP300 bromodomain inhibitor binds to a bromodomain of EP300. In some embodiments, the CBP/EP300 bromodomain inhibitor binds to one or more residues of the amino acid sequence of SEQ ID NO:4. In some embodiments, the CBP/EP300 bromodomain inhibitor binds to one or more residues of the amino acid sequence of SEQ ID NO:6. In some embodiments, the CBP/EP300 bromodomain inhibitor binds to the bromodomain of EP300 and the bromodomain of CBP. In some embodiments, the CBP/EP300 bromodomain inhibitor binds SEQ ID NO:5 and SEQ ID NO:6. In some embodiments, the CBP/EP300 bromodomain inhibitor binds SEQ ID NO:3 and SEQ ID NO:4. In some embodiments, the CBP/EP300 bromodomain inhibitor inhibits and/or reduces binding of the CBP/EP300 bromodomain to chromatin. In some embodiments, the CBP/EP300 bromodomain inhibitor does not inhibit histone acetyl transferase activity of CBP/EP300.

In some embodiments of any of the methods, the CD8 T cells in the individual have enhanced priming, activation, proliferation, and/or cytolytic activity relative to prior to the administration of the CBP/EP300 bromodomain inhibitor. In some embodiments, the number of CD8 T cells is elevated relative to prior to administration of the CBP/EP300 bromodomain inhibitors. In some embodiments, the CD8 T cells have reduced levels of expression of one or more of the following biomarkers: IFNA17, IGF1, FSCN1, SUMO2, C1orf129, EIF2S2, TDGF1, AIDA, CCR4, CD160, MC4R, KRTAP2-2, MT1JP, OR4N2, KRTAP4-5, MT1L//MT1L, IL13, LCE1D, KIR2DL2, LOC158696, LIF, IL28A, TAS2R13, CTLA4, and/or FOXP3 relative to prior to administration of the CBP/EP300 bromodomain inhibitor. In some embodiments, the CD8 T cells have reduced levels of expression of CD160 and/or KIR2DL2 relative to prior to administration of the CBP/EP300 bromodomain inhibitor.

In some embodiments of any of the methods, the enhanced immune function is characterized by Treg cells in the individual (e.g., at the tumor site(s)) have reduced levels of expression of one or more of the following markers: IL28A, GPR87, ANKRD37, CABLES1, RAPGEF2, TRIM69, MT1L//MT1L, FAM113B, FOXP3, CSF2, OCM2, GLIPR1, FGFBP2, CTLA4, CST7, GOLGA6L1, IFIT3, FAM13A, APOD, AK2, CLDN1, HSD11B1, DNAJC12, PHEX, IL2, FOXD4L3, GNA15, ZBTB32, RDH10, OR52E5, CYP2A6, GZMH, CCL20, ADM, LOC100131541, RNF122, FAM36A, AMY2B, GPR183, MYOF, IL29, AIDA, SPRY1, ENOPH1, IL1RN, SLAMF1, PGM2L1, SSBP3, MMP23B, HIST1H3J, MYO1B, BEND5, S1PR1, CDK6, GPR56, ZC3H12A, DOK5, DUSP1, CYB5R2, KCNAB2, LAG3, KLF10, GK, SHC4, IL12RB2, CD109, HAVCR2 (TIM-3), LTA, FAM40B, HMGCS1, HSPA1A, ZNF705A, CMAH, KIF3A, CHN1, KBTBD8, TNF, MOP-1, RASGRP4, INSIG1, SLAMF7, OR10H4, LPL, HIST1H2BJ, LIF, IGF1, IL18RAP, OR52N4, OR1D2, CCR4, CXCR5, IL1R1, MICAL2, NRN1, PICALM, B3GNT5, IF144L, CXCR3, ICOS, IFIT2, NCR3, HSPA1B, CD80, GNG2, C7orf68, GPR171, RPS10P7, IL23A, LOC283174, PLK2, EMP1, FNBP1L, CD226, RBMS3, IL23R, PTGER4, GZMB, F5, and/or HIST1H2BK relative to prior to administration of CBP/EP300 bromodomain inhibitor. In some embodiments, the Treg cell biomarker is one or more of LAG3, CTLA4, and/or FOXP3.

In some embodiments of any of the methods, the enhanced immune function is characterized by enhanced naïve T cell responsiveness to CD3/CD28 stimulation in the presence of Treg cells.

In some embodiments, the CD8 T cell priming is characterized by increased T cell proliferation and/or enhanced cytolytic activity in CD8 T cells. In some embodiments, the CD8 T cell activation is characterized by an elevated frequency of γ-IFN⁺ CD8 T cells. In some embodiments, the CD8 T cell is an antigen-specific T-cell. In some embodiments, the immune evasion is inhibited.

The methods provided herein are useful in treating conditions where enhanced immunogenicity is desired such as increasing tumor immunogenicity for the treatment of cancer. For example, provided herein are CBP/EP300 bromodomain inhibitors for use to enhance T-cell function to upregulate cell-mediated immune responses and for the treatment of T cell dysfunctional disorders, tumor immunity. In some embodiments, the CBP/EP300 bromodomain inhibitors promote anti-tumor immunity by inhibiting the suppressive function of regulatory T (Treg) cells and/or relieving T cell exhaustion on chronically stimulated CD8⁺ T cells.

CBP/EP300 bromodomain inhibitors are further useful in reducing Foxp3 expression during extra-thymic Treg cell differentiation. Continual Foxp3 expression is essential to maintain suppressive activity in Treg cells. In some embodiments, reduced Foxp3 expression through CBP/EP300 bromodomain inhibition impairs Treg cells suppressive activity and promote tumor anti-immunity. Treg cells are highly enriched in tumors derived from multiple cancer indications, including melanoma, NSCLC, renal, ovarian, colon, pancreatic, hepatocellular, and breast cancer. In a subset of these indications, increased intratumoral Treg cell densities are associated with poor patient prognosis. These indications include NSCLC, ovarian, pancreatic, hepatocellular, and breast cancer. CBP/EP300 bromodomain inhibitors are predicted to impair intratumoral Treg cell function in these cancer indications to enhance effector T cell activity.

T cell exhaustion is characterized by chronic CD8⁺ T cell stimulation in the absence of antigen clearance. Compared to naïve or activated effector T cells, exhausted T cells are refractory to T cell receptor stimulation due to increased expression of inhibitory receptors including PD-1, LAG-3, and TIM-3. Antagonist antibodies that block these inhibitory receptors relieve T cell suppression, thereby promote tumor cell killing. CBP/EP300 bromodomain inhibitors reduce the expression of the inhibitory receptors LAG-3 and TIM-3.

Another embodiment includes a method of increasing efficacy of a cancer treatment (e.g., cancer treatment comprising a second therapeutic agent) in an individual comprising administering to the individual an effective amount of a CBP/EP300 bromodomain inhibitor.

Another embodiment includes a method of extending the duration of response to a cancer therapy (e.g., a second therapeutic agent) in an individual, comprising administering to an individual undergoing the cancer therapy a CBP/EP300 bromodomain inhibitor, wherein the duration of response to the cancer therapy when the CBP/EP300 bromodomain inhibitor or the pharmaceutically acceptable salt thereof is administered is extended over the duration of response to the cancer therapy in the absence of the administration of the CBP/EP300 bromodomain inhibitor or the pharmaceutically acceptable salt thereof.

Another embodiment includes a method of treating cancer in an individual comprising administering to the individual (a) a CBP/EP300 bromodomain inhibitor and (b) one or more second therapeutic agent. Further provided herein methods of extending the duration of response in an individual with cancer comprising administering to the individual (a) an effective amount of a CBP/EP300 bromodomain inhibitor and (b) an effective amount of one or more second therapeutic agent. In some embodiments, the second therapeutic agent is a cytotoxic agent and/or chemotherapeutic agent. In some embodiments, the CBP/EP300 bromodomain inhibitor and the second therapeutic agent is concomitantly administered. In certain embodiments, the CBP/EP300 bromodomain inhibitor is administered prior to and/or concurrently with the one or more second therapeutic agent. In some embodiments, the CBP/EP300 bromodomain inhibitor and the second therapeutic agent is coadministered. In some embodiments, the CBP/EP300 bromodomain inhibitor and the second therapeutic agent are coformulated.

In some embodiments of any of the methods of combination therapy, the one or more second therapeutic agent is one or more of alemtuzumab, dronabinol, daclizumab, mitoxantrone, xaliproden hydrochloride, fampridine, glatiramer acetate, natalizumab, sinnabidol, immunokine NNS03, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, an anti-IL-6 receptor antibody, neurovax, pirfenidone allotrap 1258 (RDP-1258), sTNF-R1, talampanel, teriflunomide, TGF-beta2, tiplimotide, a VLA-4 antagonist (e.g. TR-14035, VLA4 Ultrahaler, or Antegran-ELAN/Biogen), an interferon gamma antagonist, or an IL-4 agonist.

In some embodiments of any of the methods of combination therapy, the one or more second therapeutic agent is a T cell signaling inhibitor (e.g. a tyrosine kinase inhibitor), or a molecule that targets T cell activation (e.g. CTLA-4-IgG, an anti-B7 family antibody, or an anti-PD-1 family antibody). For example, a method of treating or delaying progression of cancer in an individual comprising administering an effective amount of a CBP/EP300 bromodomain inhibitor and a molecule that targets T cell activation. Additionally, provided are methods of enhancing immune function in an individual having cancer comprising administering to the individual an effective amount of a CBP/EP300 bromodomain inhibitor and an effective amount of a molecule that targets T cell activation. In some embodiments, the CBP/EP300 bromodomain inhibitor or pharmaceutically acceptable salt thereof and the second therapeutic agent is concomitantly administered. In some embodiments, the CBP/EP300 bromodomain inhibitor or pharmaceutically acceptable salt thereof and the second therapeutic agent is coadministered. In certain embodiments, the CBP/EP300 bromodomain inhibitor is administered prior to and/or concurrently with the one or more second therapeutic agent. In some embodiments, the CBP/EP300 bromodomain inhibitor or pharmaceutically acceptable salt thereof and the second therapeutic agent are coformulated.

For example, provided are methods of using CBP/EP300 bromodomain inhibitors to treat and/or delay progression of cancer in combination with a PD-1 axis binding antagonist. Further provided herein are methods of enhancing immune function in an individual having cancer comprising administering to the individual an effective amount of a CBP/EP300 bromodomain inhibitor and an effective amount of a PD-1 axis binding antagonist. A PD-1 axis binding antagonist includes a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2. In some embodiments, the PD-1 binding antagonist is a molecule that inhibits the binding of PD-1 to its ligand binding partners. In a specific aspect the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342. In some embodiments, the anti-PD-1 antibody is nivolumab (CAS Registry Number: 946414-94-4). In some embodiments, the cancer is melanoma, NSCLC, and renal cell carcinoma.

In some embodiments of any of the methods of combination therapy, the one or more second therapeutic agent is an IL-11 antibody, an anti-cytokine antibody (e.g. fonotolizumab (anti-IFNg antibody)), or an anti-receptor receptor antibodies (e.g. an anti-IL-6 receptor antibody or an antibody to a B-cell surface molecule).

In some embodiments of any of the methods of combination therapy, the one or more second therapeutic agent is one or more of LJP 394 (abetimus), an agent that depletes or inactivates B-cells (e.g. Rituximab (anti-CD20 antibody) or lymphostat-B (anti-BlyS antibody)), a TNF antagonist (e.g. an anti-TNF antibody), D2E7 (adalimumab), CA2 (infliximab), CDP 571, a TNFR-Ig construct, (p75TNFRigG (etanercept), or p55TNFRigG (LENERCEPT™).

In some embodiments of any of the methods of combination therapy, the one or more second therapeutic agent is a targeted therapy. In certain embodiments, the targeted therapy is one or more of an EGFR antagonist, RAF inhibitor, and/or PI3K inhibitor.

In certain embodiments of any of the methods, the targeted therapy is an EGFR antagonist. In certain embodiments of any of the methods, the EGFR antagonist is N-(3-ethynylphenyl)-6,7-bis(2-methoxyethoxy)-4-quinazolinamine and/or a pharmaceutical acceptable salt thereof. In certain embodiments, the EGFR antagonist is N-(3-ethynylphenyl)-6,7-bis(2-methoxyethoxy)-4-quinazolinamine. In certain embodiments, the EGFR antagonist is N-(4-(3-fluorobenzyloxy)-3-chlorophenyl)-6-(5-((2-(methylsulfonyl)ethylamino)methyl)furan-2-yl)quinazolin-4-amine,di4-methylbenzenesulfonate (e.g., lapatinib). In certain embodiments of any of the methods, targeted therapy is a RAF inhibitor. In certain embodiments, the RAF inhibitor is a BRAF inhibitor. In certain embodiments, the RAF inhibitor is a CRAF inhibitor. In certain embodiments, the BRAF inhibitor is vemurafenib. In certain embodiments, the RAF inhibitor is 3-(2-cyanopropan-2-yl)-N-(4-methyl-3-(3-methyl-4-oxo-3,4-dihydroquinazolin-6-ylamino)phenyl)benzamide (e.g., AZ628 (CAS#878739-06-1)). In certain embodiments of any of the methods, the targeted therapy is a PI3K inhibitor.

In some embodiments of any of the methods of combination therapy, the one or more second therapeutic agent is a taxane. In certain embodiments, the taxane is paclitaxel. In certain embodiments, the taxane is docetaxel. In some embodiments of any of the methods of combination therapy, the one or more second therapeutic agent is a platinum agent. In certain embodiments, the platinum agent is carboplatin. In certain embodiments, the platinum agent is cisplatin. In certain embodiments of any of the methods, the cytotoxic agent is a taxane and a platinum agent. In certain embodiments, the taxane is paclitaxel. In certain embodiments, the taxane is docetaxel. In certain embodiments, the platinum agent is carboplatin. In certain embodiments, the platinum agent is cisplatin. In some embodiments of any of the methods of combination therapy, the one or more second therapeutic agent is a vinca alkyloid. In certain embodiments, the vinca alkyloid is vinorelbine. In certain embodiments of any of the methods, the chemotherapy is a nucleoside analog. In certain embodiments, the nucleoside analog is gemcitabine. In some embodiments of any of the methods of combination therapy, the one or more second therapeutic agent is radiotherapy.

In certain embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In some embodiments of any of the methods, the cancer has elevated levels of T-cell infiltration. In some embodiments of any of the methods, the cancer is associated with increased intratumoral Treg cell density. In some embodiments of any of the methods, the cancer expresses elevated levels of one or more of the following biomarkers: IL28A, GPR87, ANKRD37, CABLES1, RAPGEF2, TRIM69, MT1L//MT1L, FAM113B, FOXP3, CSF2, OCM2, GLIPR1, FGFBP2, CTLA4, CST7, GOLGA6L1, IFIT3, FAM13A, APOD, AK2, CLDN1, HSD11B1, DNAJC12, PHEX, IL2, FOXD4L3, GNA15, ZBTB32, RDH10, OR52E5, CYP2A6, GZMH, CCL20, ADM, LOC100131541, RNF122, FAM36A, AMY2B, GPR183, MYOF, IL29, AIDA, SPRY1, ENOPH1, IL1RN, SLAMF1, PGM2L1, SSBP3, MMP23B, HIST1H3J, MYO1B, BEND5, S1PR1, CDK6, GPR56, ZC3H12A, DOK5, DUSP1, CYB5R2, KCNAB2, LAG3, KLF10, GK, SHC4, IL12RB2, CD109, HAVCR2 (TIM-3), LTA, FAM40B, HMGCS1, HSPA1A, ZNF705A, CMAH, KIF3A, CHN1, KBTBD8, TNF, MOP-1, RASGRP4, INSIG1, SLAMF7, OR10H4, LPL, HIST1H2BJ, LIF, IGF1, IL18RAP, OR52N4, OR1D2, CCR4, CXCR5, IL1R1, MICAL2, NRN1, PICALM, B3GNT5, 1F144L, CXCR3, ICOS, IFIT2, NCR3, HSPA1B, CD80, GNG2, C7orf68, GPR171, RPS10P7, 1L23A, LOC283174, PLK2, EMP1, FNBP1L, CD226, RBMS3, IL23R, PTGER4, GZMB, F5, and/or HIST1H2BK compared to a reference. In some embodiments of any of the methods, the cancer expresses elevated levels of one or more of LAG3, CTLA4, and/or FOXP3 compared to a reference. In some embodiments of any of the methods, the cancer expresses elevated levels of one or more of the following biomarkers: IFNA17, IGF1, FSCN1, SUMO2, C1orf129, EIF2S2, TDGF1, AIDA, CCR4, CD160, MC4R, KRTAP2-2, MT1JP, OR4N2, KRTAP4-5, MT1L//MT1L, IL13, LCE1D, KIR2DL2, LOC158696, LIF, IL28A, TAS2R13, CTLA4, and/or FOXP3 compared to a reference. In some embodiments of any of the methods, the cancer comprises CD8 cells wherein the CD8 cells express elevated levels of one or more of the following biomarkers: IFNA17, IGF1, FSCN1, SUMO2, C1orf129, EIF2S2, TDGF1, AIDA, CCR4, CD160, MC4R, KRTAP2-2, MT1JP, OR4N2, KRTAP4-5, MT1L//MT1L, IL13, LCE1D, KIR2DL2, LOC158696, LIF, IL28A, TAS2R13, CTLA4, and/or FOXP3 compared to a reference. In some embodiments of any of the methods, the cancer comprises CD8 cells wherein the CD8 cells express elevated levels of CD160 and/or KIR2DL2 compared to a reference. In some embodiments of any of the methods, the reference is a cells or tissues with known expression levels of the biomarker of interest. In some embodiments of any of the methods, the tissue is cancer tissue with low levels of T cell infiltration and/or low intratumoral Treg cell density.

Examples of CBP/EP300 bromodomain-mediated disorders include cancers, including, but not limited, to acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies off-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In certain embodiments of any of the methods, the cancer is lung cancer, breast cancer, pancreatic cancer, colorectal cancer, and/or melanoma. In certain embodiments, the cancer is lung. In certain embodiments, the lung cancer is NSCLC. In certain embodiments, the cancer is breast cancer. In certain embodiments, the cancer is melanoma.

Presence and/or expression levels/amount of a biomarker can be determined qualitatively and/or quantitatively based on any suitable criterion known in the art, including but not limited to DNA, mRNA, cDNA, proteins, protein fragments and/or gene copy number. In certain embodiments, presence and/or expression levels/amount of a biomarker in a first sample is increased as compared to presence/absence and/or expression levels/amount in a second sample. In certain embodiments, presence/absence and/or expression levels/amount of a biomarker in a first sample is decreased as compared to presence and/or expression levels/amount in a second sample. In certain embodiments, the second sample is a reference sample, reference cell, reference tissue, control sample, control cell, or control tissue. Additional disclosures for determining presence/absence and/or expression levels/amount of a gene are described herein.

In some embodiments of any of the methods, elevated expression refers to an overall increase of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5 fold, about 1.75 fold, about 2 fold, about 2.25 fold, about 2.5 fold, about 2.75 fold, about 3.0 fold, or about 3.25 fold as compared to a reference sample, reference cell, reference tissue, control sample, control cell, control tissue, or internal control (e.g., housekeeping gene).

In some embodiments of any of the methods, reduced expression refers to an overall reduction of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

Presence and/or expression level/amount of various biomarkers in a sample can be analyzed by a number of methodologies, many of which are known in the art and understood by the skilled artisan, including, but not limited to, immunohistochemical (“IHC”), Western blot analysis, immunoprecipitation, molecular binding assays, ELISA, ELIFA, fluorescence activated cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

The amount of both the CBP/EP300 bromodomain inhibitor or salt thereof and additional agent (in those compositions which comprise an additional therapeutic agent as described above) that may be combined with the carrier materials to produce a single dosage form will vary depending upon the host treated and the particular mode of administration. In certain embodiments, compositions of this invention are formulated such that a dosage of between 0.01-100 mg/kg body weight/day of an inventive can be administered.

The additional therapeutic agent and the CBP/EP300 bromodomain inhibitor may act synergistically. Therefore, the amount of additional therapeutic agent in such compositions may be less than that required in a monotherapy utilizing only that therapeutic agent, or there may be fewer side effects for the patient given that a lower dose is used. In certain embodiments, in such compositions a dosage of between 0.01-1,000 μg/kg body weight/day of the additional therapeutic agent can be administered.

CBP/EP300 Bromodomain Inhibitors

It has been discovered that certain compounds are CBP/EP300 bromodomain inhibitors that bind specifically to the bromodomain motifs harbored in one or more of CBP and/or EP300.

In some embodiments, the CBP/EP300 bromodomain inhibitor binds to a bromodomain of CBP. In some embodiments, the CBP/EP300 bromodomain inhibitor binds to one or more residues of the amino acid sequence of SEQ ID NO:5. In some embodiments, the CBP/EP300 bromodomain inhibitor binds to one or more residues of the amino acid sequence of SEQ ID NO:3. In some embodiments, the CBP/EP300 bromodomain inhibitor binds to a bromodomain of EP300. In some embodiments, the CBP/EP300 bromodomain inhibitor binds to one or more residues of the amino acid sequence of SEQ ID NO:6. In some embodiments, the CBP/EP300 bromodomain inhibitor binds to one or more residues of the amino acid sequence of SEQ 1D NO:4. In some embodiments, the CBP/EP300 bromodomain inhibitor binds to the bromodomain of EP300 and the bromodomain of CBP. In some embodiments, the CBP/EP300 bromodomain inhibitor binds SEQ ID NO:5 and SEQ ID NO:6. In some embodiments, the CBP/EP300 bromodomain inhibitor binds SEQ 1D NO:3 and SEQ ID NO:4. In some embodiments, the CBP/EP300 bromodomain inhibitor binds to at least one (e.g., 1, 2, 3, 4, 5, 6, 7, 8, 9, 10, 11, 12 or 13) of the following CBP residues: LEU 1109, PRO 1110, PHE 1111, VAL 1115, LEU 1120, ILE 1122, TYR 1125, ALA 1164, TYR 1167, ASN 1168, ARG 1173, VAL 1174 or PHE 1177. In some embodiments, the CBP/EP300 bromodomain inhibitor binds to at least one (e.g., 1, 2, 3, 4, 5, 6, 7, 8, 9, 10, 11, 12 or 13) of the following EP300 residues: LEU 1073, PRO 1074, PHE 1075, VAL 1079, LEU 1084, ILE 1086, TYR 1089, ALA 1128, TYR 1131, ASN 1132, ARG 1137, VAL 1138 or TYR 1141.

In some embodiments, the CBP/EP300 bromodomain inhibitor interferes with the associating of CBP and/or EP300 with histones, in particular acetylated lysines in histones. In some embodiments, the CBP/EP300 bromodomain inhibitor inhibits binding of CBP and/or EP300 to chromatin (e.g., histone associated DNA). In some embodiments, the CBP/EP300 bromodomain inhibitor inhibits and/or reduces binding of the CBP bromodomain and/or EP300 bromodomain to chromatin (e.g., histone associated DNA). In some embodiments, the CBP/EP300 bromodomain inhibitor does not affect association of other domains of CBP and/or EP300 to chromatin. In some embodiments, CBP/EP300 bromodomain inhibitor binds to the CBP and/or EP300 primarily (e.g., solely) through contacts and/or interactions with the CBP bromodomain and/or EP300 bromodomain. In some embodiments, CBP/EP300 bromodomain inhibitor binds to the CBP and/or EP300 through contacts and/or interactions with the CBP bromodomain and/or EP300 bromodomain as well as additional CBP and/or EP300 residues and/or domains. Methods of assaying association with chromatin are known in the art and include, but are not limited to, chromatin fractionation, BRET assay (Promega), FRAP assay, Chromatin Immunoprecipitation (ChIP), biophysical binding assay, and/or Histone Association Assay. See, e.g., Das et al., BioTechniques 37:961-969 (2004).

In some embodiments, the CBP/EP300 bromodomain inhibitor does not affect effector function in CD8 cells (i.e., effector function is substantially the same in the presence and/or absence of the CBP/EP300 bromodomain inhibitor). In some embodiments, the CBP/EP300 bromodomain inhibitor does not affect expression levels of perforin, granzyme, and/or EOMES (i.e., expression levels of one or more perforin, granzyme, and/or EOMES are substantially the same in the presence and/or absence of the CBP/EP300 bromodomain inhibitor). In some embodiments, the CBP/EP300 bromodomain inhibitor does not affect expression levels of effector cytokines IFN-γ and/or TNFα (i.e., expression levels of effector cytokines IFN-γ and/or TNFα are substantially the same in the presence and/or absence of the CBP/EP300 bromodomain inhibitor). In some embodiments, the CBP/EP300 bromodomain inhibitor enhances naïve T cell responsiveness to CD3/CD28 stimulation in the presence of Treg cells.

In some embodiments, the CBP/EP300 bromodomain inhibitor does not substantially bind to (e.g., does not bind to) the HAT domain of CBP and/or EP300. In some embodiments, the CBP/EP300 bromodomain inhibitor does not substantially bind to (e.g., does not bind to) the HAT domain of CBP and/or EP300 as identified in Delvecchio et al., Nat. Struct. & Mol. Biol. 20:1040-1046 (2013), which is incorporated by reference in its entirety. In some embodiments, the CBP/EP300 bromodomain inhibitor does not substantially bind to one or more residues of the amino acid sequence SEQ ID NO:8 (amino acid residues 1321-1701 of UniProt No. Q92793). In some embodiments, the CBP/EP300 bromodomain inhibitor does not substantially bind to one or more residues of the amino acid sequence SEQ ID NO:7 (amino acid residues 1285-1664 of UniProt No. Q09472). In some embodiments, the CBP/EP300 bromodomain inhibitor does not inhibit the histone acetyltransferase (HAT) catalytic activity of CBP and/or EP300.

Compounds that are CBP/EP300 bromodomain inhibitors are expected to have improved and/or distinct properties over other compounds, such as “HAT” inhibitor compounds. HAT inhibition is expected to result in a global reduction in protein acetylation (histone and non-histone), likely affecting cell viability in a significant way. In some embodiments, CBP/EP300 bromodomain inhibition preserves the HAT activity of these proteins while resulting in the reduction of transcriptional activity of a relatively small subset of target genes, as shown in Table 2 and Table 3 (244 genes in Treg cells and 25 genes in CD8 cells reduced 2-fold or more).

In some embodiments, the CBP and/or EP300 inhibitor inhibits transcriptional transactivation at target regulatory sites. In some embodiments, the CBP/EP300 bromodomain inhibition eliminates or diminishes binding of CBP and/or EP300 at one or more target sites in Treg cells and CD8 cells. In some embodiments, the target site in Treg cells and CD8 cells is one or more of IL28A, GPR87, ANKRD37, CABLES1, RAPGEF2, TRIM69, MT1L//MT1L, FAM113B, FOXP3, CSF2, OCM2, GLIPR1, FGFBP2, CTLA4, CST7, GOLGA6L1, IFIT3, FAM13A, APOD, AK2, CLDN1, HSD11B1, DNAJC12, PHEX, IL2, FOXD4L3, GNA15, ZBTB32, RDH10, OR52E5, CYP2A6, GZMH, CCL20, ADM, LOC100131541, RNF122, FAM36A, AMY2B, GPR183, MYOF, IL29, AIDA, SPRY1, ENOPH1, IL1RN, SLAMF1, PGM2L1, SSBP3, MMP23B, HIST1H3J, MYO1B, BEND5, S1PR1, CDK6, GPR56, ZC3H12A, DOK5, DUSP1, CYB5R2, KCNAB2, LAG3, KLF10, GK, SHC4, IL12RB2, CD109, HAVCR2 (TIM-3), LTA, FAM40B, HMGCS1, HSPA1A, ZNF705A, CMAH, KIF3A, CHN1, KBTBD8, TNF, MOP-1, RASGRP4, INSIG1, SLAMF7, OR10H4, LPL, HIST1H2BJ, LIF, IGF1, IL18RAP, OR52N4, OR1D2, CCR4, CXCR5, IL1R1, MICAL2, NRN1, PICALM, B3GNT5, IF144L, CXCR3, ICOS, IFIT2, NCR3, HSPA1B, CD80, GNG2, C7orf68, GPR171, RPS10P7, IL23A, LOC283174, PLK2, EMP1, FNBP1L, CD226, RBMS3, IL23R, PTGER4, GZMB, F5, HIST1H2BK, IFNA17, IGF1, FSCN1, SUMO2, C1orf129, EIF2S2, TDGF1, AIDA, CCR4, CD160, MC4R, KRTAP2-2, MT1JP, OR4N2, KRTAP4-5, IL13, LCE1D, KIR2DL2, LOC158696, IL28A, and/or TAS2R13 loci. In some embodiments, the target site is one or more of FOXP3, LAG3, TIM3 and CTLA4 loci. In some embodiments, the CBP/EP300 bromodomain inhibitor inhibits CBP and/or EP300-mediated acetylation of FOXP3 by reducing binding of CBP and/or EP300 at FOXP3 and does not affect histone acetyltransferase catalytic activity.

Descriptions of CBP and EP300 (also known as p300) can be found, e.g., in Chrivia et al., Nature, 365, 855 (1993) and Teufel et al., PNAS, 104, 7009 (2007). Examples of CBP/EP300 bromodomain inhibitor compounds that may be useful in the practice of certain embodiments include compounds of Formula I, an isomer or a mixture of isomers thereof (e.g., enantiomers) or a pharmaceutically acceptable salt, solvate or prodrug thereof. Such compounds, and processes and intermediates that are useful for preparing such compounds, are described in Angew. Chem. Int. Ed., 2014, v53, pages 1-6 and corresponding supporting information. Such compounds bind to the bromodomain of CBP/EP300, forming a cation-π interaction with R1173 residue of the CBP bromodomain.

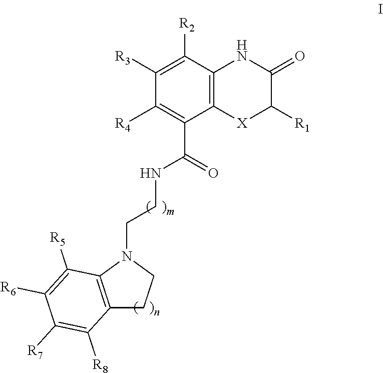

wherein:

X is NH or O;

m is 1 or 2;

n is 1 or 2;

R₁ is independently selected from the group consisting of substituted or unsubstituted C₁-C₆ alkyl, substituted or unsubstituted C₂₋₆alkenyl, substituted or unsubstituted C₂₋₆alkynyl, and substituted or unsubstituted C₃₋₆ carbocyclyl;

R₂ is independently selected from the group consisting of hydrogen, halogen, substituted or unsubstituted C₁-C₆ alkyl, substituted or unsubstituted C₂₋₆alkenyl, and substituted or unsubstituted C₂₋₆ alkynyl;

R₃ independently selected from the group consisting of hydrogen, halogen, substituted or unsubstituted C₁-C₆ alkyl, substituted or unsubstituted C₂₋₆alkenyl, and substituted or unsubstituted C₂₋₆ alkynyl;

R₄ independently selected from the group consisting of hydrogen, halogen, substituted or unsubstituted C₁-C₆ alkyl, substituted or unsubstituted C₂₋₆alkenyl, and substituted or unsubstituted C₂₋₆ alkynyl;

R₅ independently selected from the group consisting of hydrogen, halogen, substituted or unsubstituted C₁-C₆ alkyl, substituted or unsubstituted C₂₋₆alkenyl, substituted or unsubstituted C₂-6alkynyl, and OC₁-C₆ alkyl;

R6 independently selected from the group consisting of hydrogen, halogen, substituted or unsubstituted C₁-C₆ alkyl, substituted or unsubstituted C₂₋₆ alkenyl, substituted or unsubstituted C₂-6alkynyl, and OC₁-C₆ alkyl;

R7 independently selected from the group consisting of hydrogen, halogen, substituted or unsubstituted C₁-C₆ alkyl, substituted or unsubstituted C₂₋₆alkenyl, substituted or unsubstituted C₂-6alkynyl, and OC₁-C₆ alkyl; and

R₈ independently selected from the group consisting of hydrogen, halogen, substituted or unsubstituted C₁-C₆ alkyl, substituted or unsubstituted C₂₋₆ alkenyl, substituted or unsubstituted C₂₋₆alkynyl, and OC₁-C₆ alkyl;

or a salt thereof.

In certain embodiments, the compound of Formula I is selected from the group consisting of:

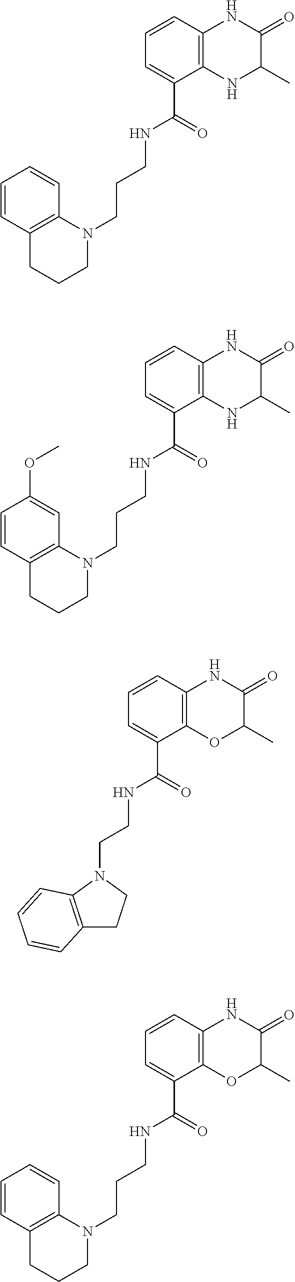 [SMILES: CC1Nc2c(cccc2C(=O)NCCCN2CCCc3ccccc32)NC1=O ; COc1ccc2c(c1)N(CCCNC(=O)c1cccc3c1NC(C)C(=O)N3)CCC2 ; CC1Oc2c(cccc2C(=O)NCCN2CCc3ccccc32)NC1=O ; CC1Oc2c(cccc2C(=O)NCCCN2CCCc3ccccc32)NC1=O]

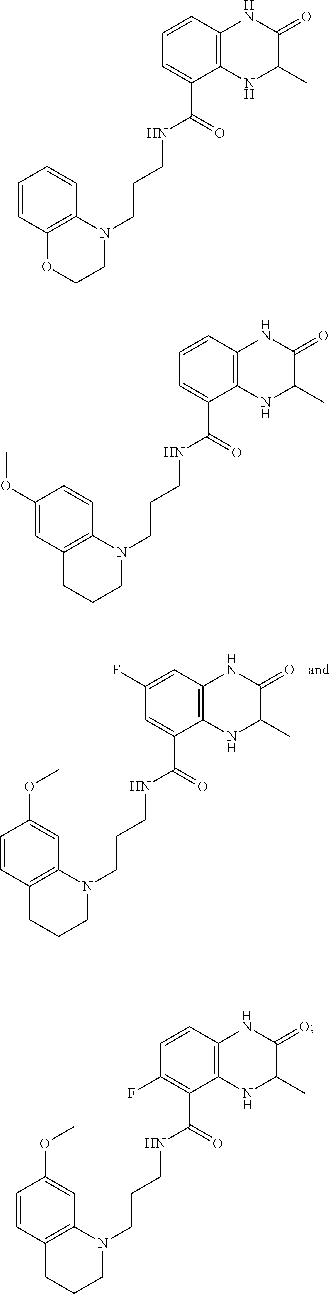 [SMILES: CC1Nc2c(cccc2C(=O)NCCCN2CCOc3ccccc32)NC1=O ; COc1ccc2c(c1)CCCN2CCCNC(=O)c1cccc2c1NC(C)C(=O)N2 ; COc1ccc2c(c1)N(CCCNC(=O)c1cc(F)cc3c1NC(C)C(=O)N3)CCC2 ; COc1ccc2c(c1)N(CCCNC(=O)c1c(F)ccc3c1NC(C)C(=O)N3)CCC2]

or a salt thereof.

In certain embodiments, the methods and uses of the present invention exclude all of these compounds:

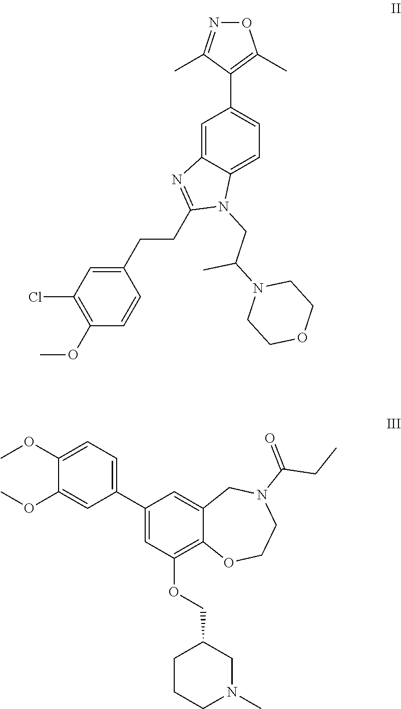 [SMILES: COc1ccc(CCc2nc3cc(-c4c(C)noc4C)ccc3n2CC(C)N2CCOCC2)cc1Cl ; CCC(=O)N1CCOc2c(cc(-c3ccc(OC)c(OC)c3)cc2OC[C@H]2CCCN(C)C2)C1]

Pharmaceutical Compositions and Methods of Administration

Further provided herein are pharmaceutical compositions comprising a CBP/EP300 bromodomain inhibitor for use in the methods described herein. In one embodiment, the composition further comprises a pharmaceutically acceptable carrier, adjuvant, or vehicle. In another embodiment, the composition further comprises an amount of the compound effective to measurably inhibit a CBP/EP300 bromodomain. In certain embodiments, the composition is formulated for administration to a patient in need thereof.

Compositions comprising a CBP/EP300 bromodomain inhibitor or salt thereof may be administered orally, parenterally, by inhalation spray, topically, transdermally, rectally, nasally, buccally, sublingually, vaginally, intraperitoneal, intrapulmonary, intradermal, epidural or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

In one embodiment, the composition comprising a CBP/EP300 bromodomain inhibitor or salt thereof is formulated as a solid dosage form for oral administration. Solid dosage forms for oral administration include capsules, tablets, pills, powders, and granules. In certain embodiments, the solid oral dosage form comprising a CBP/EP300 bromodomain inhibitor or a salt thereof further comprises one or more of (i) an inert, pharmaceutically acceptable excipient or carrier, such as sodium citrate or dicalcium phosphate, and (ii) filler or extender such as starches, lactose, sucrose, glucose, mannitol, or silicic acid, (iii) binders such as carboxymethylcellulose, alginates, gelatin, polyvinylpyrrolidinone, sucrose or acacia, (iv) humectants such as glycerol, (v) disintegrating agent such as agar, calcium carbonate, potato or tapioca starch, alginic acid, certain silicates or sodium carbonate, (vi) solution retarding agents such as paraffin, (vii) absorption accelerators such as quaternary ammonium salts, (viii) a wetting agent such as cetyl alcohol or glycerol monostearate, (ix) absorbent such as kaolin or bentonite clay, and (x) lubricant such as talc, calcium stearate, magnesium stearate, polyethylene glycols or sodium lauryl sulfate. In certain embodiments, the solid oral dosage form is formulated as capsules, tablets or pills. In certain embodiments, the solid oral dosage form further comprises buffering agents. In certain embodiments, such compositions for solid oral dosage forms may be formulated as fillers in soft and hard-filled gelatin capsules comprising one or more excipients such as lactose or milk sugar, polyethylene glycols and the like.

In certain embodiments, tablets, dragees, capsules, pills and granules of the compositions comprising a CBP/EP300 bromodomain inhibitor or salt thereof optionally comprise coatings or shells such as enteric coatings. They may optionally comprise opacifying agents and can also be of a composition that they release the active ingredient(s) only, or preferentially, in a certain part of the intestinal tract, optionally, in a delayed manner. Examples of embedding compositions include polymeric substances and waxes, which may also be employed as fillers in soft and hard-filled gelatin capsules using such excipients as lactose or milk sugar as well as high molecular weight polethylene glycols and the like.

In another embodiment, a composition comprises a micro-encapsulated CBP/EP300 bromodomain inhibitor or salt thereof, and optionally, further comprises one or more excipients.

In another embodiment, compositions comprise liquid dosage formulations comprising a CBP/EP300 bromodomain inhibitor or salt thereof for oral administration, and optionally further comprise one or more of pharmaceutically acceptable emulsions, microemulsions, solutions, suspensions, syrups and elixirs. In certain embodiments, the liquid dosage form optionally, further comprise one or more of an inert diluent such as water or other solvent, a solubilizing agent, and an emulsifier such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols or fatty acid esters of sorbitan, and mixtures thereof. In certain embodiments, liquid oral compositions optionally further comprise one or more adjuvant, such as a wetting agent, a suspending agent, a sweetening agent, a flavoring agent and a perfuming agent.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a CBP/EP300 bromodomain inhibitor, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In certain embodiments, the composition for rectal or vaginal administration are formulated as suppositories which can be prepared by mixing a CBP/EP300 bromodomain inhibitor or a salt thereof with suitable non-irritating excipients or carriers such as cocoa butter, polyethylene glycol or a suppository wax, for example those which are solid at ambient temperature but liquid at body temperature and therefore melt in the rectum or vaginal cavity and release the CBP/EP300 bromodomain inhibitor.

Example dosage forms for topical or transdermal administration of a CBP/EP300 bromodomain inhibitor include ointments, pastes, creams, lotions, gels, powders, solutions, sprays, inhalants or patches. The CBP/EP300 bromodomain inhibitor or a salt thereof is admixed under sterile conditions with a pharmaceutically acceptable carrier, and optionally preservatives or buffers. Additional formulation examples include an ophthalmic formulation, ear drops, eye drops, transdermal patches. Transdermal dosage forms can be made by dissolving or dispensing the CBP/EP300 bromodomain inhibitor or a salt thereof in medium, for example ethanol or dimethylsulfoxide. Absorption enhancers can also be used to increase the flux of the compound across the skin. The rate can be controlled by either providing a rate controlling membrane or by dispersing the compound in a polymer matrix or gel.

Nasal aerosol or inhalation formulations of a CBP/EP300 bromodomain inhibitor or a salt thereof may be prepared as solutions in saline, employing benzyl alcohol or other suitable preservatives, absorption promotors to enhance bioavailability, fluorocarbons, and/or other conventional solubilizing or dispersing agents.

In certain embodiments, pharmaceutical compositions may be administered with or without food. In certain embodiments, pharmaceutically acceptable compositions are administered without food. In certain embodiments, pharmaceutically acceptable compositions of this invention are administered with food.

Specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided CBP/EP300 bromodomain inhibitor or salt thereof in the composition will also depend upon the particular compound in the composition.

In one embodiment, the effective amount of the compound of the invention administered parenterally per dose will be in the range of about 0.01-100 mg/kg, alternatively about 0.1 to 20 mg/kg of patient body weight per day, with the typical initial range of compound used being 0.3 to 15 mg/kg/day. In another embodiment, oral unit dosage forms, such as tablets and capsules, contain from about 5 to about 100 mg of the compound of the invention.

An example tablet oral dosage form comprises about 2 mg, 5 mg, 25 mg, 50 mg, 100 mg, 250 mg or 500 mg of a CBP/EP300 bromodomain inhibitor or salt thereof, and further comprises about 5-30 mg anhydrous lactose, about 5-40 mg sodium croscarmellose, about 5-30 mg polyvinylpyrrolidone (PVP) K30 and about 1-10 mg magnesium stearate. The process of formulating the tablet comprises mixing the powdered ingredients together and further mixing with a solution of the PVP. The resulting composition can be dried, granulated, mixed with the magnesium stearate and compressed to tablet form using conventional equipment. An example of an aerosol formulation can be prepared by dissolving about 2-500 mg of a compound of formula I or salt thereof, in a suitable buffer solution, e.g. a phosphate buffer, and adding a tonicifier, e.g. a salt such sodium chloride, if desired. The solution may be filtered, e.g. using a 0.2 micron filter, to remove impurities and contaminants.

The CBP/EP300 bromodomain inhibitors or salts thereof may be employed alone or in combination with other agents for treatment as described above. For example, the second agent of the pharmaceutical combination formulation or dosing regimen may have complementary activities to the CBP/EP300 bromodomain inhibitor such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a CBP/EP300 bromodomain inhibitor or a salt thereof, and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Typically, any agent that has activity against a disease or condition being treated may be co-administered. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

### EXAMPLES

The following are examples of methods and compositions of the invention. It is understood that various other embodiments may be practiced, given the general description provided above.

### Example 1

### CBP/EP300 as a Small Molecule Target for Cancer Immunotherapy

To discover how CBP/EP300 bromodomains might be targets for the treatment of cancer, the functional impact of using potent and selective small molecule inhibitor compounds designed to bind to CBP/EP300 bromodomains was investigated, thus preventing their association with acetylated histones in chromatin. Since small molecule inhibitors can have off-target effects, a panel of compounds from distinct chemical scaffolds with a range of biochemical potencies (active compounds, Table 1) was tested to rule out such off-target effects. Furthermore, compounds sharing the same scaffolds as the active compounds, but with no activity against the bromodomains of CBP/EP300 (inactive compounds, Table 1) were used as negative controls.

TABLE 1

| COMPOUND | POTENCY (IC50, uM) | |
|---|---|---|
| CBP/EP300(1) | 0.5 | Active |
| CBP/EP300(2) | 0.27 | Active |
| CBP/EP300(A) | >20 | Inactive |
| CBP/EP300(B) | >20 | Inactive |

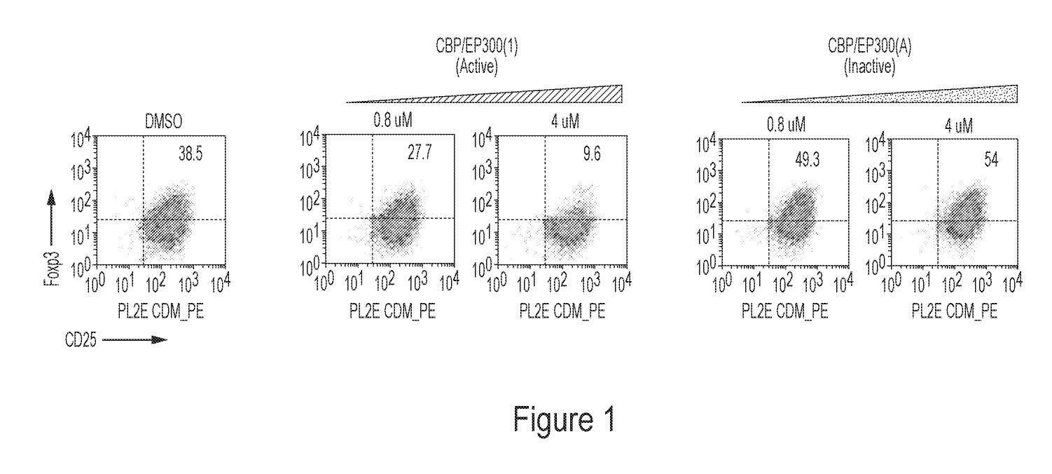

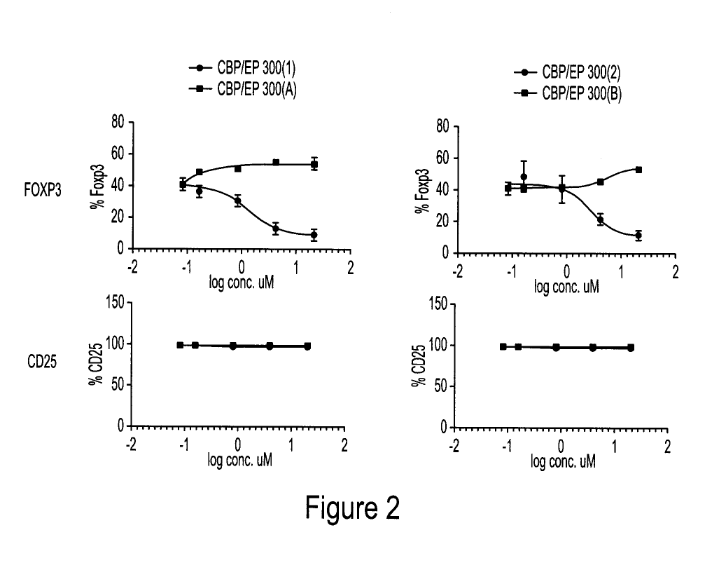

In a first set of experiments, Treg cells from purified naïve human CD4+ T cells were prepared. These naïve T cells can be identified by their surface expression of the marker CD45RA, and then differentiated in vitro into Treg cells with a standard and well established mix of cytokines, as described in the Methods section. Treg cells can be readily identified by their expression of FOXP3, a transcription factor that is necessary for the differentiation and function of these cells (Josefowicz et al., Immunity, 30, 616-625 (2009)). Human naïve T cells were cultured under Treg-differentiating conditions in the presence of active compound targeting the bromodomains of CBP/EP300, or inactive control compound. As shown in FIG. 1, the CBP/EP300 inhibitor CBP/EP300(1), but not the inactive compound CBP/EP300(A), was shown to reduce the number of FOXP3+ cells generated in these experiments, as seen by flow cytometry. These observations were confirmed and expanded by producing dose-response curves under the same culture conditions described above with two exemplar active compounds from distinct chemical scaffolds, CBP/EP300(1) and CBP/EP300(2). These active compounds, but not the inactive ones, CBP/EP300(A) and CBP/EP300(B), did reduce the number of FOXP3+ cells in a dose-dependent manner (FIG. 2, upper panels). Importantly, the activation marker CD25 was not affected by any compound treatment, suggesting that these cells are functional, although unable to differentiate into the Treg lineage (FIG. 2, lower panels). From these sets of experiments, it was concluded that that CBP/EP300 bromodomain inhibition results in an impairment of naïve T cells to differentiate into Treg cells.

The impact of CBP/EP300 bromodomain inhibition in Treg cell gene expression was further investigated. With that aim, full-genome transcription profiling was performed, comparing samples from cultures under the same conditions as those described in FIG. 1, incubated with active compound CBP/EP300(1), or DMSO (compound vehicle, control). As an additional control, naïve T cells were cultured in the absence of differentiating cytokines, hereafter described as TH0 (see Methods section). 244 genes were down-modulated 2-fold or more at the transcript level. The down-regulated genes include FOXP3 (as predicted from the data shown in FIG. 1 and FIG. 2), but also other genes that are thought to play important roles in Treg cell function, such as LAG3, TIM3 and CTLA4. From these results, it was concluded that CPB/EP300 bromodomain inhibition results in the suppression of a network of genes that largely define Treg cells and their biological functions, including suppression of proliferation of conventional T cells.

TABLE 2
CBP/EP300 bromodomain inhibition results in a 2 or more fold reduction
of transcriptional activity of 244 genes in Treg cells

| | | | | | |
|---|---|---|---|---|---|
| IL28A | GPR87 | ANKRD37 | CABLES1 | RAPGEF2 | TRIM69 |
| MT1L // | FAM113B | FOXP3 | CSF2 | OCM2 | GLIPR1 |
| MT1L | | | | | |
| FGFBP2 | CTLA4 | CST7 | GOLGA6L1 | IFIT3 | FAM13A |
| APOD | AK2 | CLDN1 | HSD11B1 | DNAJC12 | PHEX |
| IL2 | FOXD4L3 | GNA15 | ZBTB32 | RDH10 | OR52E5 |
| CYP2A6 | GZMH | CCL20 | ADM | LOC100131541 | RNF122 |
| FAM36A | AMY2B | GPR183 | MYOF | IL29 | AIDA |
| SPRY1 | ENOPH1 | IL1RN | SLAMF1 | PGM2L1 | SSBP3 |
| MMP23B | HIST1H3J | MYO1B | BEND5 | S1PR1 | CDK6 |
| GPR56 | ZC3H12A | DOK5 | DUSP1 | CYB5R2 | KCNAB2 |
| LAG3 | KLF10 | GK | SHC4 | IL12RB2 | CD109 |
| HAVCR2 | LTA | FAM40B | HMGCS1 | HSPA1A | ZNF705A |
| (TIM-3) | | | | | |
| CMAH | KIF3A | CHN1 | KBTBD8 | TNF | MOP-1 |
| RASGRP4 | INSIG1 | SLAMF7 | OR10H4 | LPL | HIST1H2BJ |
| LIF | IGF1 | IL18RAP | OR52N4 | OR1D2 | CCR4 |
| CXCR5 | IL1R1 | MICAL2 | NRN1 | PICALM | B3GNT5 |
| IFI44L | CXCR3 | ICOS | IFIT2 | NCR3 | HSPA1B |
| CD80 | GNG2 | C7orf68 | GPR171 | RPS10P7 | IL23A |
| LOC283174 | PLK2 | EMP1 | FNBP1L | CD226 | RBMS3 |
| IL23R | PTGER4 | GZMB | F5 | HIST1H2BK | |

TABLE 3
CBP/EP300 bromodomain inhibition results in a 2 or more fold reduction
of transcriptional activity of 25 genes in CD8 cells

| | | | | |
|---|---|---|---|---|
| IFNA17 | IGF1 | FSCN1 | SUMO2 | C1orf129 |
| EIF2S2 | TDGF1 | AIDA | CCR4 | |
| CD160 | MC4R | KRTAP2-2 | MT1JP | |
| OR4N2 | KRTAP4-5 | MT1L // | IL13 | |
| | | MT1L | | |
| LCE1D | KIR2DL2 | LOC158696 | LIF | |
| IL28A | TAS2R13 | CTLA4 | FOXP3 | |

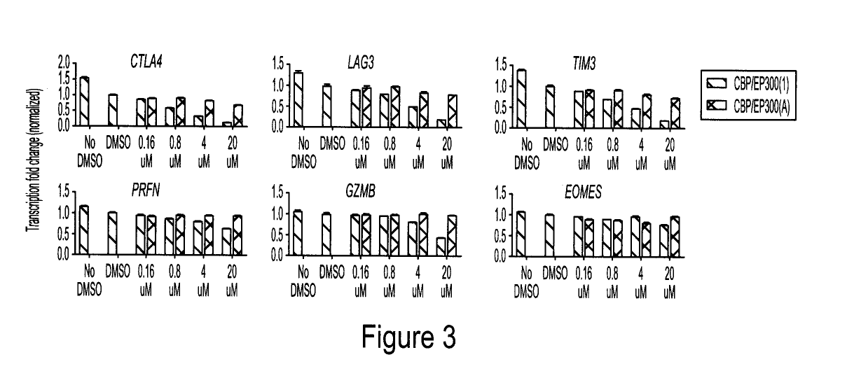

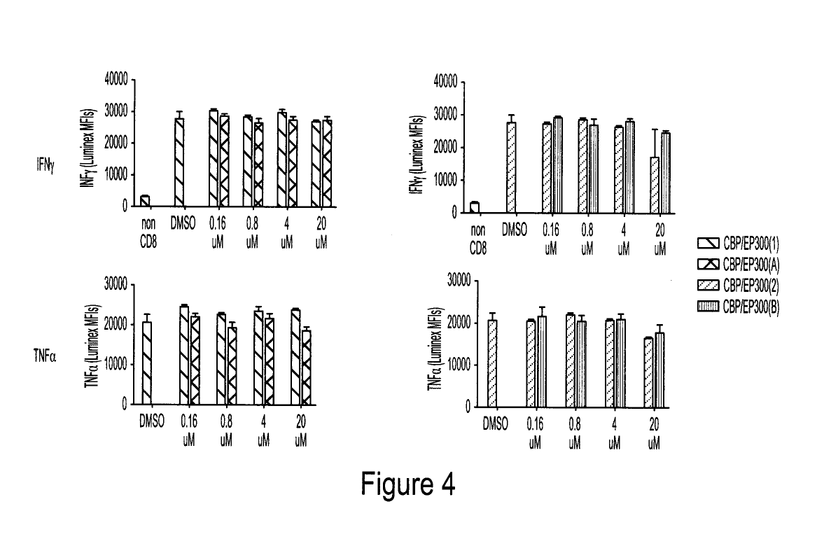

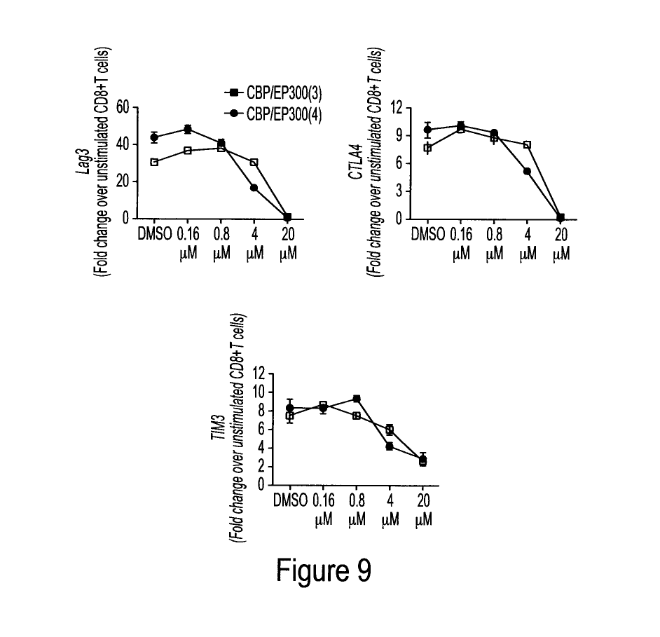

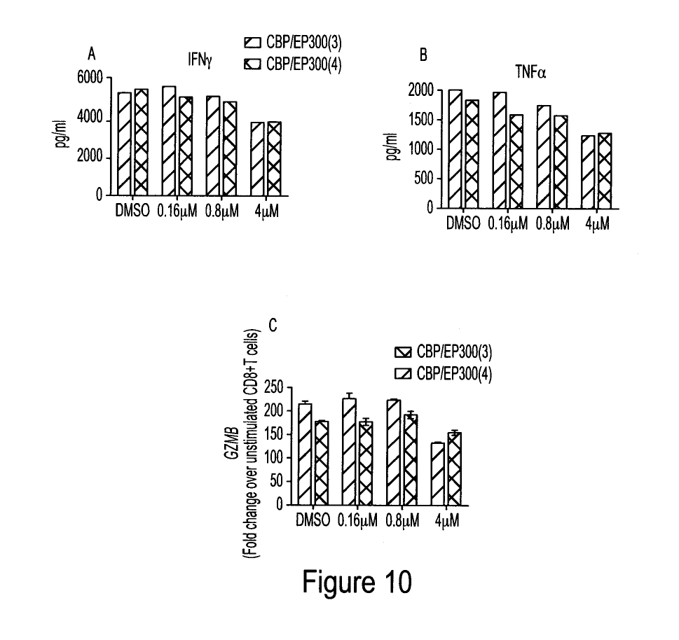

One major mechanism of evasion of the immune system by cancer cells is known as T cell exhaustion. In this state, cancer cells induce in T cells, and especially in CD8+ T cells, a transcriptional state that makes these cells unresponsive and unable to exert cytotoxic functions. A key characteristic of this process is the expression of inhibitory receptors on the surface of these CD8 cells, such as PD-1, LAG3, TIM3 and CTLA4 (Wherry, Nat. Immunol., 12, 492-499 (2011). Because, as described herein, it was discovered that LAG3, TIM3 and CTLA4 are under the transcriptional control of CBP/EP300 bromodomains, whether CBP/EP300 bromodomain inhibition also resulted in the suppression of those genes in CD8 cells was investigated as a method to inhibit their expression with CBP/EP300 inhibitors and thereby reverse CD8 exhaustion. As shown in FIG. 3 (upper panels), incubation of human CD8 cells with CBP/EP300(1), but not with the inactive compound, CBP/EP300(A), resulted in a dose-dependent reduction in the expression of LAG3, TIM3 and CTLA4. Similarly, as shown in FIG. 9, CBP/EP300(3) and CBP/EP300(4) both result in a dose-dependent reduction in the expression of LAG3, TIM3 and CTLA4. Interestingly, CBP/EP300 bromodomain inhibition with CBP/EP300(1) did not affect effector function in CD8 cells, as the genes encoding Perforin, Granzyme B and EOMES (FIG. 3, lower panels) were not significantly changed upon compound treatment. Furthermore, production of the effector cytokines IFN-γ and TNFα (FIG. 4) were not affected by compound treatment. Similar trends were observed for CBP/EP300(3) and CBP/EP300(4) as shown in FIG. 10. Moreover, whole genome transcriptional analysis of CD8 cells upon treatment with CBP/EP300(1) revealed that additional genes involved in exhaustion, such as CD160 and KIR2DL2, were also reduced. From these results, it was concluded that CBP/EP300 bromodomain inhibition results in the selective blockade of key inhibitory receptors that are important in the regulation of CD 8 cells exhaustion.

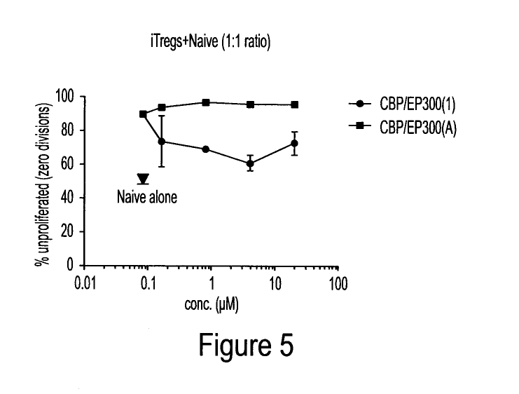

In order to investigate if the effects of CBP/EP300 bromodomain inhibition on Treg cells resulted in a functional impairment of these cells to suppress proliferation of conventional T cells, suppression assays combining Tregs and CFSE-labeled naïve T cells were carried out. Proliferation of naïve T cells was monitored in these studies by FACS-based quantification of the dye, CFSE, as it gets diluted with each cell division cycle. As shown in FIG. 5, ˜50% of naïve T cells were able to proliferate upon CD3/CD28 stimulation in the absence of Treg cells. However, when naïve T cells were combined with Treg cells, less than 10% were able to proliferate. Incubation with CBP/EP300(1) resulted in a dose-dependent inhibition of the Treg suppressive capacity, as seen by a corresponding increase in the percentage of naïve T cells able to proliferate. The inactive compound, CBP/EP300(A) had no impact, demonstrating specificity.

In summary, CBP/EP300 bromodomains play unexpected but critical roles in Treg cells and in CD8+ T cells. CBP/EP300 bromodomains control the differentiation of Treg cells and the expression of critical genes that control key biological functions in Treg cells. Additionally, CBP/EP300 bromodomain inhibition results in an impairment of the suppressive ability of Treg cells. In CD8+ T cells, CBP/EP300 bromodomains control a subset of genes that includes important ones that control exhaustion. Therefore, by coordinately suppressing Treg function and reversing CD8+ T cell exhaustion, CBP/EP300 bromodomain inhibition is beneficial in the treatment of human cancers by cancer immunotherapy.

Methods

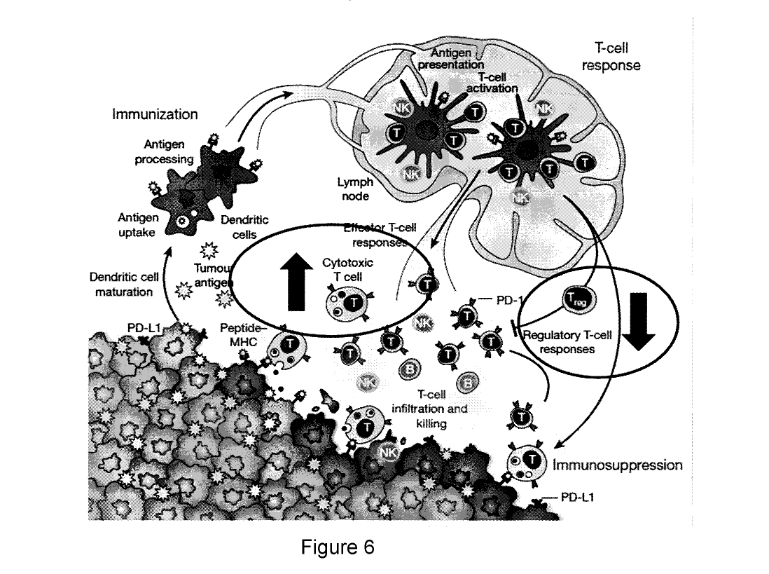

Methods for Data Presented in FIGS. 1-6

Human T Cell Cultures:

Naive CD4+CD45RA+ T cells were isolated from healthy human donor leukopaks to a purity >95% using Miltenyi naive human T cell isolation kits (Cat #130-094-131). Isolated cells were cultured at 10̂6 cells/mL under iTreg-polarizing conditions, using human T activator Dynabeads at a 1:1 ratio of beads to cells (Invitrogen; Cat#11132D), human TGFβ at 10 ng/mL and human IL-2 at 10 U/mL (R&D Cat#100-B and 202-IL, respectively). Compounds were added 16 h post-activation; final concentration of 0.5% DMSO in culture. For “unpolarized” Th0 cultures, isolated cells were cultured with Dynabeads alone, without the addition of exogenous cytokines. CD8 T cells were isolated from healthy human donor leukopaks using the Miltenyi human CD8 T cell isolation kits (Cat#130-095-236) and cultured at 10″6 cells/mL with human T activator Dynabeads at a 1:1 ratio of beads to cells, in the presence of 100 U/mL human IL-2.

FACS:

Cells from the iTreg cultures were first stained with CD25:PE (eBioscience; Cat#12-0259-42); this was followed by a fixation/permeabilization step and staining for intracellular FOXP3 using a human FOXP3 staining kit (eBioscience; Cat#77-5774-40) according to the manufacturer's protocol. FOXP3 expression by FACS was typically measured 4 d post-activation.

Expression Analysis:

RNA was isolated using the RNeasy Plus kit (Qiagen; Cat #74136). This was followed by cDNA synthesis and qPCR using Taqman primers and probes (Invitrogen). Reactions were run in duplicate or triplicate, and results analyzed by the deldelCT method, normalizing against DMSO control; Glucose-6-Phosphate Dehydrogenase (G6PD) was used as house-keeping gene (Roche; Cat#05 046 246 001). For global transcriptional profiling, samples were processed and hybridized on Affymetrix exon arrays, and data was acquired, at ALMAC Diagnostics. CEL files were processed with the RMA algorithm on core probe sets using Affymetrix′ Expression Console program. Duplicate log 2 expression values were averaged and subtracted to obtain log fold change. For the heat maps, genes having at least 2-fold change and an unadjusted Student's T-test p-value <0.10 was selected.

Suppression Assay:

iTreg cells were cultured for 84 h as described and added at a 1:1 ratio with naive CD4 T cells which had been stained with CFSE (Molecular Probes; Cat# C34554; manufacturer's protocol). Cells were activated using Dynabeads in a final volume of 200 uL in 96-well round-bottomed plates. Compounds were added 16 h post-activation at a final concentration of 0.5% DMSO in culture. Proliferation, as assayed by dilution of CFSE and appearance of lower intensity peaks, was measured at 60 h post-activation.

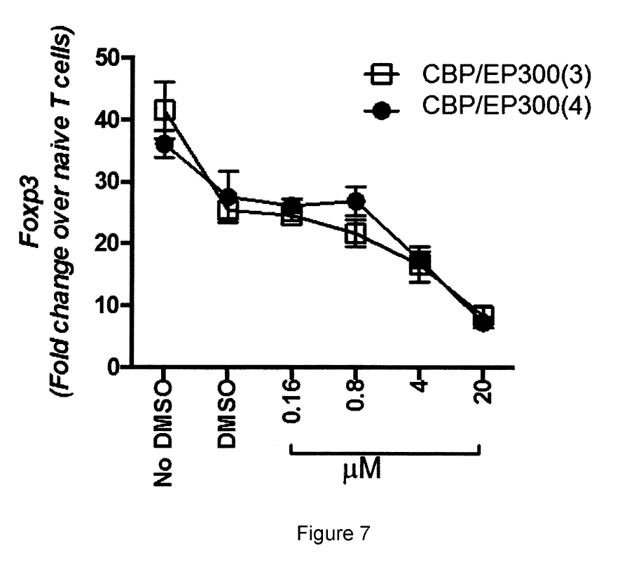

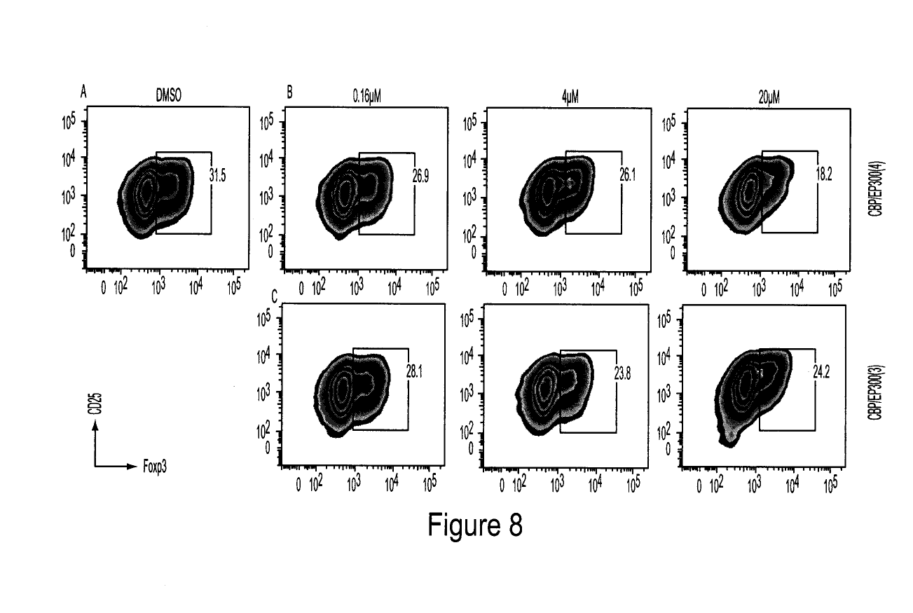

Methods for Data Presented in FIGS. 7-10

Human T Cell Cultures:

Naïve CD4+CD45RA+ T cells were isolated from healthy human PBMCs using Miltenyi Biotec naïve human T cell isolation kit (cat#130-094-131). Isolated cells were cultured at 10e6 cells/ml under iTreg-differentiation conditions using Dynabeads (Invitrogen; cat#11132D) at 1:1 ratio of beads to cells+human recombinant IL-2 (10 ng/ml) (R&D, cat#202-IL-010)+human recombinant TGFb (10 ng/ml) (Invitrogen; cat# PHG9204). After 16 hours the CBP inhibitors CBP/EP300(3) and CBP/EP300(4) were added using DMSO as control. CD8+ T cells were isolated from healthy human PBMCs using Militenyi Biotec human CD8 T cell isolation kit (cat#130-095-236) and cultured at 10e6 cells/ml with human T activator Dynabeads at 1:1 ratio of cells to cells with the addition of 10 ng/ml of recombinant human IL-2. 3 days after CD8+ T cell stimulation supernatants were collected and analyzed for CD8+ T cell associated effector function cytokines IFNg and TNFα by Luminex. Data in FIG. 10 show IFNγ (A) and TNFα (B) (pg/ml) secreted in the supernatants of CD8+ T cells stimulated with compounds CBP/EP300(3) and CBP/EP300(4), using DMSO as control. CBP inhibitors minimally affect cytokine production by CD8+ T cells.

FACS:

Cells from iTreg cultures were first stained with CD4 APC-CY7 and CD25 Pacific blue (both from BD pharmigen, cat#557811 and 560355, respectively); this was followed by fixation/permeabilization step and staining for intracellular Foxp3 FITC using human Foxp3 staining kit (eBioscience; cat#77-5774-40) according to the manufacturer protocol. FOXP3 expression by FACS was typically measured 4 d post-activation.

Expression Analysis:

3 days after CD8+ T cell stimulation mRNA was extracted using mRNA Catcher™ PLUS Purification Kit (Invitrogen; K1570-02). Gene expression of Lag3, CTLA4 and TIM3, genes encoding inhibitory receptors on the surface of CD8+ T cells and under the transcriptional control of CBP/p300, was analyzed by q-RT-PCR. Foxp3 expression and Granzyme B (GZMB) expression, a gene encoding effector function of CD8+ T cells, were also analyzed by q-RT-PCR. Beta-2-microglobulin (B2M) was used as house-keeping gene. CBP inhibitors resulted in a dose-dependent reduction in the expression of Lag3, CTLA4 and TIM3.

CBP/EP300 Bromodomain Inhibitors:

CBP/EP300(3) has the following structure:

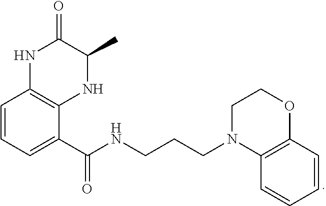 [SMILES: C[C@H]1Nc2c(cccc2C(=O)NCCCN2CCOc3ccccc32)NC1=O]

CBP/EP300(4) has the following structure:

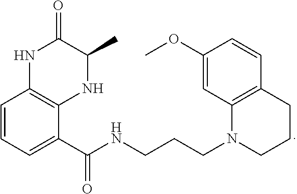 [SMILES: COc1ccc2c(c1)N(CCCNC(=O)c1cccc3c1N[C@H](C)C(=O)N3)CCC2]

In addition to the order detailed herein, the methods described herein can be performed in any suitable order unless otherwise indicated herein or otherwise clearly contradicted by context. The use of any and all examples, or exemplary language (e.g., “such as”) provided herein, is intended merely to better illuminate embodiments of invention and does not necessarily impose a limitation on the scope of the invention unless otherwise specifically recited in the claims. No language in the specification should be construed as indicating that any non-claimed element is essential to the practice of the invention.

All documents cited herein are incorporated by reference.

While a number of embodiments have been described, these examples may be altered to provide other embodiments that utilize the compounds and methods described herein. Therefore, the scope of this invention is to be defined by the appended claims rather than by the specific embodiments that have been represented by way of example.

1. A method for treating or delaying progression of cancer in an individual comprising administering an effective amount of a CBP/EP300 bromodomain inhibitor to the individual.

2. A method of enhancing immune function in an individual having cancer comprising administering an effective amount of a CBP/EP300 bromodomain inhibitor to the individual.

3. The method of claim 1, wherein CD8 T cells in the individual have enhanced priming, activation, proliferation and/or cytolytic activity relative to prior to the administration of the CBP/EP300 bromodomain inhibitor.

4. The method of claim 3, wherein the number of CD8 T cells is elevated relative to prior to administration of the CBP/EP300 bromodomain inhibitor.

5. The method of claim 3, wherein the CD8 T cell is an antigen-specific CD8 T cell.

6. The method of claim 1, wherein the cancer has elevated levels of T-cell infiltration.

7. The method of claim 1, wherein the cancer is associated with increased intratumoral Treg cell density.

8. The method of claim 1, wherein the cancer is selected from acoustic neuroma, acute leukemia, acute lymphocytic leukemia, acute myelocytic leukemia, acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer (NSCLC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

9. The method of claim 1, wherein the cancer is melanoma, NSCLC, renal, ovarian, colon, pancreatic, hepatocellular, or breast cancer.

10. The method of claim 1, wherein the cancer is NSCLC, ovarian, pancreatic, hepatocellular, or breast cancer.

11. The method of claim 1, wherein the cancer is melanoma, NSCLC, or renal cell carcinoma.

12. The method of claim 1, wherein the CBP/EP300 bromodomain inhibitor inhibits CBP.

13. The method of claim 1, wherein the CBP/EP300 bromodomain inhibitor inhibits EP300.

14. The method of claim 1, wherein the method suppresses Treg function.

15. The method of claim 1, wherein the method decreases T cell exhaustion of CD8⁺ T cells.

16. The method of claim 1, wherein the CBP/EP300 bromodomain inhibitor does not bind to the HAT domain of CBP and/or EP300.

17. (canceled)

18. (canceled)

19. A method for selecting an anti-cancer compound, comprising determining whether a test compound is a CBP/EP300 bromodomain inhibitor compound, wherein a test compound that is a CBP/EP300 bromodomain inhibitor compound is selected as an anti-cancer compound.

20. The method of claim 19, further comprising determining whether the test compound binds to the HAT domain of CBP and/or EP300, wherein a test compound that does not bind to the HAT domain of CBP and/or EP300 is selected as an anti-cancer compound.

21. The method of claim 19, further comprising determining whether the test compound suppresses Treg function, wherein a test compound that suppresses Treg function is selected as an anti-cancer compound.

22. The method of claim 19, further comprising determining whether the test compound decreases T cell exhaustion of CD8⁺ T cells, wherein a test compound that decreases T cell exhaustion of CD8⁺ T cells is selected as an anti-cancer compound.
